(19) United States
(12) Patent Application Publication
(10) Pub. No.: US 2010/0227908 A1
(43) Pub. Date: Sep. 9, 2010

(54) DIAGNOSTIC, PROGNOSTIC AND TREATMENT METHODS

(75) Inventor: MURRAY JOHN CAIRNS, WARATAH NSW (AU)

Correspondence Address: KNOBBE MARTENS OLSON & BEAR LLP, 2040 MAIN STREET, FOURTEENTH FLOOR, IRVINE, CA, 92614, US

(73) Assignee: NEWCASTLE INNOVATION LIMITED, CALLAGHAN NSW (AU)

(21) Appl. No.: 12/564,848

(22) Filed: Sep. 22, 2009

Related U.S. Application Data
(60) Provisional application No. 61/157,849, filed on Mar. 5, 2009.

(51) Int. Cl. A61K 31/7088; C12Q 1/68

(52) U.S. Cl. 514/44 A; 435/6; 514/44 R

(57) ABSTRACT

The present invention relates generally to diagnostic and prognostic protocols for schizophrenia and its manifestations including sub-threshold phenotypes and states thereof. Profiling and stratifying individuals for schizophrenia and its various manifestations also form part of the present invention as well as monitoring and predicting efficacy of therapeutic, psychiatric, social or environmental intervention. The present invention further contemplates methods of treatment of schizophrenia and symptoms thereof.

### RELATED APPLICATIONS

This application claims the benefit of U.S. Provisional Application No. 61/157,849 filed Mar. 5, 2009, which is hereby expressly incorporated by reference in its entirety.

### BACKGROUND OF THE INVENTION

1. Field of the Invention

The present invention relates generally to diagnostic and prognostic protocols for schizophrenia and its manifestations including sub-threshold phenotypes and states thereof. Profiling and stratifying individuals for schizophrenia and its various manifestations also form part of the present invention as well as monitoring and predicting efficacy of therapeutic, psychiatric, social or environmental intervention. The present invention further contemplates methods of treatment of schizophrenia and symptoms thereof.

2. Description of the Related Art

Reference to any prior art in this specification is not, and should not be taken as, an acknowledgment or any form of suggestion that this prior art forms part of the common general knowledge in any country.

Psychological “disorders” are endemic in many societies. Reference to “disorders” in this context means that an individual exhibits behavioral patterns which are inconsistent with societal norms. Most psychological phenotypes have both environmental and genetic risk factors and bases. Early detection of disorders using genetic technology has considerable potential to identify those at risk prior to the development of this chronic condition. Commencement of a low dose antipsychotic regime and early cognitive behavioral therapy, for example, may prevent the emergence of more debilitating symptoms. Development of the full disorder is associated with significant impairment of social, cognitive and occupational functioning.

Schizophrenia is a particularly complex psychological phenotype characterized by a diverse range and spectrum of symptoms and neurocognitive impairments. Schizophrenia is a common, chronic, disabling illness with an incidence of 15 new cases per 100,000 population per year (Kelly et al, Ir. J. Med. Sci. 172:37-40, 2003). Additionally, “unaffected” first degree relatives show both child (Niendam et al, Am. J. Psychiatry. 160:2060-2062, 2003) and adult (MacDonald et al, Arch. Gen. Psychiatry. 60:57-65, 2003) deficits in cognitive functioning. Siblings of those with schizophrenia also exhibit an abnormal MRI response in the dorsolateral prefrontal cortex (DLPFC) implicating inefficient information processing (Callicott et al, Am. Psychiatry. 160:709-719, 2003). Furthermore, individuals with schizophrenia and their unaffected siblings show both reductions in hippocampal volume and hippocampal shape deformity (Tepest et al, Biol, Psychiatry. 54:1234-1240, 2003). Decreased temporoparietal P300 amplitude and increased frontal P300 amplitude are found in both schizophrenic patients and their siblings (Winterer et al, Arch. Gen. Psychiatry. 60:1158-1167, 2003). Taken together, these findings indicate that the underlying pathophysiological state of schizophrenia is considerably more widespread in the general population than prevalence figures for schizophrenia would suggest and that a considerable genetic vulnerability for this disorder exists.

While its exact pathogenesis remains obscure, there is a broad consensus that schizophrenia is of neurodevelopmental origin, arising through the complex interplay of numerous genetic and environmental factors (Harrison Curr Opin Neurobiol 7:285-289, 1997). Some insight into molecular interactions within this matrix has been provided by high throughput gene expression analyses of post-mortem brain tissues (Mimics et al, Neuron 28:53-67, 2000; Hakak et al, Proc Natl Acad Sci USA 98:4746-4751, 2001; Weidenhofer et al, Mol Cell Neurosci 31:243-250, 2006; Bowden et al, BMC Genomics 9:199, 2008; Kim and Webster Correlation analysis between genome-wide expression profiles and cytoarchitectural abnormalities in the prefrontal cortex of psychiatric disorders, 2008). These investigations have shown consistently that the activity of a large number of genes are affected in schizophrenia. While some of these changes reflect alterations in known candidate genes and their downstream influences, most are inexplicable and their origins may lie well beyond the reach of these well known mechanisms. Despite the perplexing array of findings, there are patterns in schizophrenia-associated gene expression indicative of systematic regulatory dysfunction. Where these coincide with functional pathways, for example, in neurotransmitter systems and neural development, they support plausible hypotheses that correspond with a limited understanding of schizophrenia pathophysiology.

Efforts to understand the underlying mechanisms driving these changes in gene expression have focused predominantly on genetic and epigenetic influences on transcription, mediated by alterations in signal transduction pathways, their transcription factors, or gene promoter elements and associated chromatin structure.

There is a need to identify genetic factors predictive of a state of, or risk of developing, schizophrenia or its manifestations including sub-threshold phenotypes and states. Such genetic factors further provide targets for therapeutic intervention.

### SUMMARY OF THE INVENTION

The present invention identifies a pathophysiological link between genetic indicators in the post-transcriptional environment in cells of a subject and the manifestations of schizophrenia. The term “schizophrenia” as used herein is to be considered as an individual condition as well as a spectrum of conditions including sub-threshold phenotypes and states thereof. In particular, the present invention provides diagnostic targets in the form of expression of the DGCR8 gene, homologs thereof and associated genetic molecules such as miRNAs which, when elevated, is instructive as to the presence of schizophrenia or a predisposition thereto. In a further embodiment, the post-transcriptional environment results in down stream gene silencing. Such affected genes also are considered diagnostic and prognostic targets of schizophrenia. The genetic indicators further provide therapeutic targets for the development of medicaments in the treatment of schizophrenia and its symptoms.

In particular, an increase in global miRNA expression is associated with an elevation of primary miRNA processing and corresponds with an increase in the microprocessor component, DGCR8. The biological implications for this extensive increase in miRNA-modified gene silencing are profound and is over represented in pathways involved in synaptic plasticity and includes many genes and pathways associated with schizophrenia.

The early detection of schizophrenia and its related or associated conditions enables therapeutic, psychological, social and/or environment intervention at a point which more readily facilitates control over the disease condition. The genetic indicators herein are also useful in monitoring therapeutic protocols and for profiling or stratifying individuals or family members for schizophrenia. The genetic indicators are also therapeutic targets for medicaments which modulate expression of DGCR8, the global or individual miRNA environment or genes affected thereby.

Hence, the present invention contemplates a method for detecting a risk profile for schizophrenia or a manifestation thereof or a sub-threshold phenotype or state thereof in a subject, the method comprising identifying an elevation in expression of the DGCR8 gene or a homolog thereof or a genetic molecule associated therewith wherein an elevation in DGCR8 or its homolog or associated genetic molecule is indicative of a risk of having or developing symptoms of schizophrenia.

A genetic molecule associated with DGCR8 includes miRNA's and genetic factors such as those targeted by the primers listed in Table 3 or their families. These include hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2. In a particular embodiment, the genetic molecule associated with DGCR8 is selected from the miR-15 and miR-107 families.

Identifying a “risk profile” for schizophrenia includes identifying schizophrenia or its symptoms.

The present invention further contemplates the use of DGCR8 or a homolog thereof or a genetic molecule associated therewith in the manufacture of a diagnostic or prognostic assay for schizophrenia or a manifestation thereof or a sub-threshold phenotype or state thereof.

Global miRNA levels, and in particular miRNA or genetic factors such as hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2 as well as levels of DGCR8 expression, may be detected in a range of biological fluids or tissues. Particular target tissues include the cerebral cortex such as the superior temporal gyrus (STG) and dorsolateral prefrontal cortex (DLPFC). Other tissues or samples, include neural cells or neural fluid, stem cells, lymphocytes and other immune cells.

Methods for monitoring the therapeutic, psychological, social and environmental intervention of subjects diagnosed and/or suspected of having schizophrenia also form part of the present invention.

The present invention further provides diagnostic and prognostic kits for schizophrenia or manifestations thereof or sub-threshold phenotypes or states thereof. Such kits may be supplied generally or limited to health care providers.

The present invention also provides a method for the treatment or prophylaxis of schizophrenia or manifestations thereof in a subject, the method comprising administering an agent which down-regulates (e.g. an antagonist) the level of a molecule associated with schizophrenia or manifestations thereof.

Hence, another aspect of the present invention provides a method for the treatment or prophylaxis of schizophrenia or manifestations thereof in a subject, the method comprising administering an antagonist of expression or function of a molecule selected from hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e and FXR2, DICER, DGCR8, DROSHA, XPO5, DDX26, DDX5 and FXR2 under conditions to reduce levels of the molecule.

The antagonists of the present invention include, without being limited to, antisense oligonucleotides, antagomiRs and microRNAs sponges.

The present invention further provides therapeutic targets for the development of medicaments in the treatment of schizophrenia and its manifestations and symptoms. Therapeutic targets include miRNAs or genes or other genetic factors such as hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2. In a particular embodiment, the miRNAs are selected from the miR-15 and miR-107 families.

Hence, the present invention further provides a use of a genetic indicator of schizophrenia or its manifestations and sub-threshold phenotypes selected from DGCR8 and a genetic factor selected from hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2 or other family members thereof in the manufacture of a medicament in the amelioration of symptoms of schizophrenia.

Such medicaments include anti-sense and sense RNA species, dsRNA species, anti-miRNAs and antagomirs. Methods of treating schizophrenia and its phenotypes also form part of the present invention.

Nucleotide sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

TABLE 1
Summary of Sequence Identifiers

| SEQ ID NO. | DESCRIPTION |
|---|---|
| 1 | Nucleotide sequence of hsa-miR-15a |
| 2 | Nucleotide sequence of hsa-miR-15b |
| 3 | Nucleotide sequence of hsa-miR-195 |
| 4 | Nucleotide sequence of hsa-miR-16 |
| 5 | Nucleotide sequence of hsa-miR-107 |
| 6 | Nucleotide sequence of 3′→ 5′ HTR2A-107 MRE |
| 7 | Nucleotide sequence of 5′→ 3′ HTR2A-107 MRE |
| 8 | Primer - U6-probe |
| 9 | Primer - U6-F339 |
| 10 | Primer - U49-F |
| 11 | Primer - U49-R |
| 12 | Primer - U44-F |
| 13 | Primer - U44-R |
| 14 | Primer - 107-F |
| 15 | Primer - 107-R |
| 16 | Primer - 15a-F^{b} |
| 17 | Primer - 15a-R |
| 18 | Primer - 15b-F |
| 19 | Primer - 15b-R |
| 20 | Primer - 16-F |
| 21 | Primer - 16-R |
| 22 | Primer 128a-F |
| 23 | Primer - 128a-R |
| 24 | Primer - 181a-F |
| 25 | Primer - 181a-R |
| 26 | Primer - 181b-F |
| 27 | Primer - 181b-R |
| 28 | Primer - 195-F |
| 29 | Primer - 195-R |
| 30 | Primer - 19a-F |
| 31 | Primer - 19a-R |
| 32 | Primer - 20a-F |
| 33 | Primer - 20a-R |
| 34 | Primer - 219-F |
| 35 | Primer - 219-R |
| 36 | Primer - 26b-F |
| 37 | Primer - 26b-R |
| 38 | Primer - 27a-F |
| 39 | Primer - 27a-R |
| 40 | Primer - 29b-F |
| 41 | Primer - 29c-R |
| 42 | Primer - 338-F |
| 43 | Primer - 338-R |
| 44 | Primer - 7-F |
| 45 | Primer - 7-R |
| 46 | Primer - let-7d-F |
| 47 | Primer - let-7d-R |
| 48 | Primer - let 7e-F |
| 49 | Primer - let-7-e-R |
| 50 | Primer M13-F |
| 51 | Primer - GUSB-F |
| 52 | Primer - GUSB-R |
| 53 | Primer - HMBS-F |
| 54 | Primer - HMBS-R |
| 55 | Primer - FXR2-F |
| 56 | Primer - FXR2-R |
| 57 | Primer - DICER1-F |
| 58 | Primer - DICER-R |
| 59 | Primer - DGCR8-F |
| 60 | Primer - DGCR8-R |
| 61 | Primer - DROSHA-F |
| 62 | Primer - DROSHA-R |
| 63 | Primer - XPO5-F |
| 64 | Primer - XPO5-R |
| 65 | Primer - DDX26-F2 |
| 66 | Primer - DDX26-R2 |
| 67 | Primer - DDX5-F |
| 68 | Primer - DDX5-R |
| 69 | Primer - DDX17-F |
| 70 | Primer - DDX17-R |
| 71 | Primer - FXR2-F |
| 72 | Primer - FXR2-R |
| 73 | Primer - pri-181b-2-F1 |
| 74 | Primer - pr-181b-2-R1 |
| 75 | Primer - pre-181b-2-F |
| 76 | Primer - pre-181b-2-R |
| 77 | Primer -pri-26b-F |
| 78 | Primer - pri-26b-R |
| 79 | Primer - pre-26b-F |
| 80 | Primer - pre-26b-R |
| 81 | Cassette - VSNL1-107-T |
| 82 | Cassette - VSNL1-107-B |
| 83 | Cassette - RELN-107-T |
| 84 | Cassette - RELN-107-B |
| 85 | Cassette - HTR2A-107-T |
| 86 | Cassette - HTR2A-107-B |
| 87 | Cassette - GRIN3A-107-T |
| 88 | Cassette - GRIN3A-107-B |
| 89 | Cassette - PLEXNA2-1070T |
| 90 | Cassette - PLEXNA2-107-B |
| 91 | Cassette - DLG4-107-T |
| 92 | Cassette - DLG4-107-B |
| 93 | Cassette - DRD1-107-T |
| 94 | Cassette - DRD1-107-B |
| 95 | Cassette - GRM7-107-T |
| 96 | Cassette - GRM7-107-B |
| 97 | Cassette - RGS4-107-T |
| 98 | Cassette - RGS4-107-B |
| 99 | miRNA - miR-107⁺ |
| 100 | miRNA - miR-107⁻ |
| 101 | miRNA - miR-15a⁺ |
| 102 | miRNA - miR-15-a⁻ |
| 103 | miRNA - miR-15b⁺ |
| 104 | miRNA - miR-15b⁻ |
| 105 | miRNA - miR-16⁺ |
| 106 | miRNA - miR-16⁻ |
| 107 | miRNA - miR-195⁺ |
| 108 | miRNA - miR-195⁻ |
| 109 | miRNA - control-miRNA-1⁺ |
| 110 | miRNA - control-miRNA-1⁻ |
| 111 | miRNA - control-miRNA-2⁺ |
| 112 | miRNA - control-miRNA-2⁻ |
| 113 | Anti-miRs - anti-miR-107 |
| 114 | Anti-miRs - anti-miR-15a |
| 115 | Anti-miRs - anti-miR-15b |
| 116 | Anti-miRs - anti-miR-16 |
| 117 | Anti-miRs - anti-miR-195 |
| 118 | Anti-miRs - control anti-miR-1 |
| 119 | anti-miRs - control anti-miR-2 |

### BRIEF DESCRIPTION OF THE DRAWINGS

Some figures contain color representations or entities. Color photographs are available from the Patentee upon request or from an appropriate Patent Office. A fee may be imposed if obtained from a Patent Office.

FIGS. 1a through d are graphical representations showing schizophrenia-associated miRNA expression in the Superior temporal gyrus (STG). (a) Average fold-change of miRNA expression (schizophrenia to control) was plotted against log transformed fluorescence values (n=17 matched pairs). A global increase in miRNA expression in the STG in schizophrenia is indicated by the majority of miRNA displaying a ratio greater than 1.0. (b) Electrophoresis of dephosphorylated total RNA labeled with polynucleotide kinase (PNK). Whole lane densitometry of the phosphorimage indicated an increase in small RNA in the schizophrenia cohort (pink trace) compared to the controls (blue trace), particularly in the small RNA fraction (20-24 nt) region corresponding to most miRNA. (c) Increased miRNA expression in the STG was validated using real-time RT-PCR (n=21 matched pairs). Level of expression for controls set at 1. Bars are mean±SEM. * p<0.05; ** p<0.01; *** p<0.001. (d) Q-PCR expression data hierarchically clustered (correlation uncentered, average linkage; Cluster 3.0). Blue indicates low expression and yellow indicates high expression (Java Treeview).

FIGS. 2 a through d are graphical representations showing schizophrenia-associated miRNA expression in the dorsolateral prefrontal cortex (DLPFC). (a) miRNA expression in the DLPFC in schizophrenia was characterized by global up regulation illustrated in this scatter plot (see FIG. 1a for description) by the majority of individual miRNA displaying a ratio greater than 1.0. (b) Increased miRNA expression in the DLPFC was validated using Q-PCR (n=15 matched pairs). Level of expression for controls set at 1. (c) Further Q-PCR expression analysis indicated that 12 miRNA with altered expression displayed an up-regulation in both the STG and DLPFC. Bars indicate mean fold-change +SEM. * p<0.05; ** p<0.01; *** p<0.001. (d) Q-PCR expression data was subjected to hierarchical clustering and heat map displayed as described in FIG. 1d.

FIGS. 3a through d are graphical representations showing alterations in miRNA processing in schizophrenia. (a) Simplified schematic of miRNA biogenesis showing genes involved in key enzymatic steps. (b) Primary, precursor and mature transcripts for miR-181b were analyzed by Q-PCR in the STG. The primary transcript was not altered in schizophrenia, however the precursor and mature transcripts were both up-regulated 1.4-fold (p=0.048) and 1.7-fold (p=0.039) respectively. The host gene of miR-26b (CDTSPI) and primary transcript were not altered. The precursor and mature miR-26b transcript were both up-regulated in schizophrenia (1.5 fold (p=0.023) and 1.9-fold (p=0.001) respectively. In the DLPFC, a similar trend followed. Host gene and primary transcripts were not altered in schizophrenia. For miR181b, the precursor and mature were up-regulated 1.5-fold (p=0.043) and 1.4-fold (p=0.039) respectively. For miR-26b, the precursor and mature were up-regulated 1.6-fold (p=0.046) and 2.2-fold (p=0.001) respectively. (c) Expression of miRNA biogenesis genes was analyzed in the STG (n=21 matched pairs) and the DLPFC (n=15 matched pairs). DGCR8 was significantly up-regulated in the STG and DLPFC, and Drosha, Dicer and DDX26 were up-regulated in the DLPFC only. Bars indicate mean fold-change (schizophrenia to control)+SEM. * p<0.05; ** p<0.01 unpaired Student's t-test. (d) DGCR8 expression was determined by Q-PCR in matched paired samples (SZ Vs CTR). DGCR8 was up-regulated in 16 out of 21 matched pairs of STG tissue and in 13 out of 15 matched pairs of DLPFC tissue.

FIGS. 4a through c are schematic and graphical representations showing Regulation of schizophrenia associated reporter gene constructs by miRNA. (a) Sequence alignment showing miR-107 and the miR-15 family seed region homology (grey highlight). Together, the two groups were predicted to have many target genes in common (Venn diagram). (b) The pMIR-REPORT miRNA expression reporter system contains a firefly luciferase gene under the control of CMV promoter. Putative miRNA recognition elements for various schizophrenia candidate genes were inserted into the multiple cloning site in the 3′-UTR of the firefly luciferase gene (HTR2A shown as an example). (c) A matrix chart showing the relative activity of reporter gene constructs (x-axis) in response to co-transfected miRNA (dark bars) or their cognate anti-miR (light bars). Relative luciferase activity for each reporter/miRNA/anti-miR combination was expressed as a percentage of the response to scrambled controls (+SD; * p<0.05).

### DETAILED DESCRIPTION OF THE PREFERRED EMBODIMENT

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The singular forms “a”, “an”, and “the” include single and plural aspects unless the context clearly indicates otherwise. Thus, for example, reference to “a miRNA” includes a single miRNA, as well as two or more miRNAs; reference to “an association” includes a single association or multiple associations; reference to “the invention” includes single or multiple aspects of an invention; and so forth.

The present invention is predicated in part on the identification of an alteration and in particular a substantial alteration in the post-transcriptional environment characterized by an elevation in DGCR8 expression and a global increase in miRNA expression.

This change in post-transcriptional expression environment has implications for the development and ongoing pathophysiology of schizophrenia as each miRNA has the capacity to regulate the expression of multiple target genes. In accordance with the present invention, an association between an alteration in levels of miRNAs such as those targeted by the primers listed in Table 3 or their families or genes or other genetic factors and schizophrenia is identified. Examples of these genetic factors include hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5-F, FXR2. Particular miRNAs are family members of miR-15 and miR-107 in which expression is elevated with schizophrenia. Similarly, an association between DGCR8 expression in schizophrenia is identified. Hence, an increase in global miRNA expression corresponds to an increase in the microprocessor component, DGCR8. There is a convergent influence of global miRNA which is over represented in synaptic plasticity including genes associated with schizophrenia. Hence, the present invention extends to DGCR8 expression and global miRNA levels as well as genes silenced by the miRNA, as diagnostic and prognostic markers of schizophrenia.

Hence, the present invention contemplates a method for detecting a risk profile for schizophrenia or a manifestation thereof or a sub-threshold phenotype or state thereof in a subject, the method comprising identifying an elevation in expression of the DGCR8 gene or a homolog thereof or a genetic molecule associated therewith wherein an elevation in DGCR8 or its homolog or associated genetic molecule is indicative of a risk of having or developing symptoms of schizophrenia.

Reference to “schizophrenia” includes a condition generally described as schizophrenia or a condition having symptoms related thereto. Schizophrenia can be considered a disease with a spectrum of manifestations with various threshold levels. Symptoms of schizophrenia may appear in a range of related disorders including classical schizophrenia as well as addiction, dementia, anxiety disorders, bipolar disorder, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia.

Reference herein to “schizophrenia” includes, therefore, conditions which have symptoms similar to schizophrenia and hence are regard as schizophrenia-related conditions. Such symptoms of schizophrenia include behavioral and physiological conditions. A related condition may also have a common underlying genetic cause or association and/or a common treatment rationale. Due to the composition of schizophrenia and related conditions, the ability to identify a genetic profile or set of genetic risk factors to assist in defining schizophrenia is of significant importance. The present invention now provides this genetic profile generally within the post-transcriptional cellular environment. Furthermore, identification of potential genetic profiles may include a predisposition to developing schizophrenia or a related neurological, psychiatric or psychological condition

A “neurological, psychiatric or psychological condition, phenotype or state” may be an adverse condition or may represent “normal” behavior. The latter constitutes behavior consistent with societal “norms”.

Reference herein to a “subject” includes a human which may also be considered an individual, patient, host, recipient or target.

The present invention enables, therefore, a stratification of subjects based on a genetic profile. The genetic profile includes expression levels of DGCR8 or a homolog thereof or a genetic molecule associated therewith. The stratification or profiling enables early diagnosis, conformation of a clinical diagnosis, treatment monitoring and treatment selection for a neurological, psychiatric or psychological conditions phenotype or state.

Another aspect of the present invention contemplates a method for stratifying subjects for schizophrenia, said method comprising determining levels of expression of DGCR8 or a homolog thereof or a genetic molecule associated therewith wherein an elevation in DGCR8 or its homolog or associated genetic molecule places a subject in a group of schizophrenia or at risk schizophrenia subjects.

Yet another aspect of the present invention is directed to the use of DGCR8 or a homolog thereof or a genetic molecule associated therewith in the manufacture of a diagnostic or prognostic assay for schizophrenia or a manifestation thereof or a sub-threshold phenotype or state thereof.

A genetic molecule associated with DGCR8 includes global miRNA and other genetic factors such as or one or more of hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2. Particular genetic factors are miRNAs selected from the miR-15 and miR-107 families Furthermore, miRNAs may result in down regulation of a gene. Hence, that gene becomes a diagnostic or prognostic target.

There are many methods which may be used to detect a DGCR8 expression or mRNAs including determining the presence via sequence identification. Direct nucleotide sequencing, either manual sequencing or automated fluorescent sequencing can detect the presence of a particular mRNA species

A rapid preliminary analysis to nucleic acid species can be performed by looking at a series of Southern or Northern blots. Each blot may contain a series of “normal” individuals and a series of individuals having schizophrenia or a related neurological, psychiatric or psychological condition, phenotype or state.

Techniques for detecting nucleic acid species include PCR or other amplification technique

Nucleic acid analysis via microchip technology is also applicable to the present invention. In this technique, thousands of distinct oligonucleotide probes are built up in an array on a silicon chip. Nucleic acids to be analyzed are fluorescently labeled and hybridized to the probes on the chip. It is also possible to study nucleic acid-protein interactions using these nucleic acid microchips. Using this technique, one can determine the presence of nucleic acid species or even the level of a nucleic acid species as well as the expression levels of DGCR8. The method is one of parallel processing of many, including thousands, of probes at once and can tremendously increase the rate of analysis.

Hence, alteration of mRNA expression from a genetic loci can be detected by any techniques known in the art. These include Northern blot analysis, PCR amplification and RNase protection. Diminished mRNA expression indicates an alteration of an affected gene. Alteration of DGCR8 expression can also be detected by screening for alteration of expression product such as a protein. For example, monoclonal antibodies immunoreactive with a target DGCR8 protein can be used to screen a tissue. Lack of cognate antigen or a reduction in the levels of antigen would indicate a reduction in expression of DGCR8. Such immunological assays can be done in any convenient formats known in the art. These include Western blots, immunohistochemical assays and ELISA assays. Any means for detecting an altered protein can be used to detect alteration of the wild-type protein. Functional assays, such as protein binding determinations, can be used.

Hence, the present invention further extends to a method for identifying a genetic basis behind diagnosing or treating schizophrenia or a manifestation thereof including a sub-threshold phenotype or state, the method comprising obtaining a biological sample from an individual and detecting the level of expression of DGCR8 or homolog thereof or a genetic molecule associated therewith wherein the presence of an elevated level of DGCR8 expression or an associated genetic molecule is instructive or predictive of schizophrenia or related conditions.

The biological sample is any fluid or cell or tissue in which DGCR8 is expressed or where mRNA's have increased or where expression of another gene has been down regulated. In one embodiment, the biological sample is a biopsy from the cerebral cortex including the STG or DLPFC. In another embodiment, the biological sample is a neural cell or neural fluid, stem cell or lymphocyte or other immune cell.

The present invention identifies the presence of genetic molecules associated with schizophrenia or associated conditions or a risk of developing same. In order to detect a nucleic acid molecule a biological sample is prepared and analyzed for a difference in levels between the subject being tested and a control. In this context, a “control” includes the levels in a statistically significant normal population.

Amplification-based detection assays are particularly useful. As used herein, the phrase “amplifying” refers to increasing the content of a specific genetic region of interest within a sample. The amplification of the genetic region of interest may be performed using any method of amplification known to those of skill in the relevant art. In one aspect, the present method for detecting an mRNA species utilizes PCR as the amplification step.

PCR amplification utilizes primers to amplify a genetic region of interest. Reference herein to a “primer” is not to be taken as any limitation to structure, size or function. Reference to primers herein, includes reference to a sequence of deoxyribonucleotides comprising at least three nucleotides. Generally, the primers comprises from about three to about 100 nucleotides, preferably from about five to about 50 nucleotides and even more preferably from about 10 to about 25 nucleotides such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. The primers of the present invention may be synthetically produced by, for example, the stepwise addition of nucleotides or may be fragments, parts or portions or extension products of other nucleic acid molecules.

In an embodiment, one of the at least two primers is involved in an amplification reaction to amplify a target sequence. If this primer is also labeled with a reporter molecule, the amplification reaction will result in the incorporation of any of the label into the amplified product. The terms “amplification product” and “amplicon” may be used interchangeably.

The primers and the amplicons of the present invention may also be modified in a manner which provides either a detectable signal or aids in the purification of the amplified product.

A range of labels providing a detectable signal may be employed. The label may be associated with a primer or amplicon or it may be attached to an intermediate which subsequently binds to the primer or amplicon. The label may be selected from a group including a chromogen, a catalyst, an enzyme, a fluorophore, a luminescent molecule, a chemiluminescent molecule, a lanthanide ion such as Europium (Eu³⁴), a radioisotope and a direct visual label. In the case of a direct visual label, use may be made of a colloidal metallic or non-metallic particular, a dye particle, an enzyme or a substrate, an organic polymer, a latex particle, a liposome, or other vesicle containing a signal producing substance and the like. A large number of enzymes suitable for use as labels is disclosed in U.S. Pat. Nos. 4,366,241, 4,843,000 and 4,849,338. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme which is in solution. Alternatively, a fluorophore which may be used as a suitable label in accordance with the present invention includes, but is not limited to, fluorescein-isothiocyanate (FITC), and the fluorochrome is selected from FITC, cyanine-2, Cyanine-3, Cyanine-3.5, Cyanine-5, Cyanine-7, fluorescein, Texas red, rhodamine, lissamine and phycoerythrin.

Examples of fluorophores are provided in Table 2.

TABLE 2
Representative flurophores

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bromide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

In order to aid in the purification of an amplicon, the primers or amplicons may additionally be incorporated on a bead. The beads used in the methods of the present invention may either be magnetic beads or beads coated with streptavidin.

The extension of the hybridized primer to produce an extension product is included herein by the term amplification. Amplification generally occurs in cycles of denaturation followed by primer hybridization and extension. The present invention encompasses form about one cycle to about 120 cycles, preferably from about two to about 70 cycles, more preferably from about five to about 40 cycles, including 10, 15, 20, 25 and 30 cycles, and even more preferably, 35 cycles such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 cycles.

In order for the primers used in the methods of the present invention to anneal to a nucleic acid molecule containing the gene of interest, a suitable annealing temperature must be determined. Determination of an annealing temperature is based primarily on the genetic make-up of the primer, i.e. the number of A, T, C and Gs, and the length of the primer. Annealing temperatures contemplated by the methods of the present invention are from about 40° C. to about 80° C., preferably from about 50° C. to about 70° C., and more preferably about 65° C. such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80° C.

The PCR amplifications performed in the methods of the present invention include the use of MgCl₂ in the optimization of the PCR amplification conditions. The present invention encompasses MgCl₂ concentrations for about 0.1 to about 10 mM, preferably from 0.5 to about 5 mM, and even more preferably 2.5 mM such as 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 mM.

In one embodiment, results of nucleic acid detection tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Suitable diagnostic techniques include those described herein as well as those described in U.S. Pat. Nos. 5,837,492; 5,800,998 and 5,891,628.

The identification of the association between the pathophysiology of schizophrenia and levels of expression of DCGR8 or miRNAs permits the early presymptomatic screening of individuals to identify those at risk for developing schizophrenia or to identify the cause of such a disorder or the risk that any individual will develop same. Genetic testing enables practitioners to identify or stratify individuals at risk for certain behavioral states associated with schizophrenia or its manifestations including or an inability to overcome symptoms or schizophrenia after initial treatment. For particular at risk couples, embryos or fetuses may be tested after conception to determine the genetic likelihood of the offspring being pre-disposed to schizophrenia. Certain behavioral or therapeutic protocols may then be introduced from birth or early childhood to reduce the risk of developing schizophrenia. Presymptomatic diagnosis will enable better treatment of schizophrenia, including the use of existing medical therapies. Genotyping of individuals will be useful for (a) identifying a form of schizophrenia which will respond to particular drugs, (b) identifying a schizophrenia which responds well to specific medications or medication types with fewer adverse effects and (c) guide new drug discovery and testing.

Further, the present invention provides a method for screening drug candidates to identify molecules useful for treating schizophrenia involving a drug which affects DGCR8 expression or levels of associated genetic molecules. The terms “drug”, “agent”, “therapeutic molecule”, “prophylactic molecule”, “medicament”, “candidate molecule” or “active ingredient” may be used interchangeable in describing this aspect of the present invention. It also includes a pro-drug.

The present invention provides, therefore, information necessary for medical practitioners to select drugs for use in the treatment of schizophrenia. With the identification of a genetic risk of schizophrenia antipsychotic medications can be selected for the treatment.

Hence, the present invention contemplates the use of a genetic indicator of schizophrenia or its manifestations and sub-threshold phenotypes selected from DGCR8 and an miRNA or gene or other genetic factor selected from the listing comprising hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2 in the manufacture of a medicament in the amelioration of symptoms of schizophrenia. Such medicaments include anti-sense and sense RNA species, anti-miRNAs and antagomirs. Methods of treating schizophrenia and its phenotypes also form part of the present invention. As indicated above, particular genetic indicators are miRNAs selected from the miR-15 and miR-107 families.

The present invention also provides a method for the treatment or prophylaxis of schizophrenia or manifestations thereof in a subject, the method comprising administering an agent which down-regulates the level of a molecule associated with schizophrenia or manifestations thereof in a subject.

In one embodiment, the molecule associated with schizophrenia or manifestations thereof is DGCR8. In another embodiment, the molecules is an miRNA selected from hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e or a molecule selected from FXR2, DICER, DGCR8, DROSHA, XPO5, DDX26, DDX5 and FXR2.

Hence, another aspect of the present invention provides a method for the treatment or prophylaxis of schizophrenia or manifestations thereof in a subject, the method comprising administering an antagonist of expression or function of a molecule selected from hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e and FXR2, DICER, DGCR8, DROSHA, XPO5, DDX26, DDX5 and FXR2 for a time and under conditions to reduce levels of the molecule.

Examples of antagonists include any molecule which down regulates the expression or function of one or more DGCR8, hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e and FXR2, DICER, DGCR8, DROSHA, XPO5, DDX26, DDX5 and FXR2, including anti-sense molecules, antagomiRs and microRNAs sponges (Ebert et al. Nature Methods 4(9):721-726, 2007).

The synthesis of oligonucleotide with antagonistic activity against specific miRNA, including miRNAs hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e disclosed herein, is described Krutzfeldt et al. Nature 438:685-689, 2005.

Oligonucleotides may include modifications designed to improve their delivery into cells, their stability once inside a cell, and/or their binding to the appropriate miRNA target. For example, the oligonucleotide sequence may be modified by the addition of one or more phosphorothioate (for example phosphoromonothioate or phosphorodithioate) linkages between residues in the sequence, or the inclusion of one or morpholine rings into the backbone. Alternative non-phosphate linkages between residues include phosphonate, hydroxlamine, hydroxylhydrazinyl, amide and carbamate linkages (see, for example, United States Patent Application Publication No. 20060287260, Manoharan I., the disclosure of which is incorporated herein in its entirety), methylphosphonates, phosphorothiolates, phosphoramidates or boron derivatives. The nucleotide residues present in the oligonucleotide may be naturally occurring nucleotides or may be modified nucleotides. Suitable modified nucleotides include 2′-O-methyl nucleotides, such as 2′-O-methyl adenine, 2′-O-methyl-uracil, 2′-O-methyl-thymine, 2′-O-methyl-cytosine, 2′-O-methyl-guanine, 2′-O-methyl-2-amino-adenine; 2-amino-adenine, 2-amino-purine, inosine; propynyl nucleotides such as 5-propynyl uracil and 5-propynyl cytosine; 2-thio-thymidine; universal bases such as 5-nitro-indole; locked nucleic acid (LNA), and peptide nucleic acid (PNA). The ribose sugar moiety that occurs naturally in ribonucleosides may be replaced, for example with a hexose sugar, polycyclic heteroalkyl ring, or cyclohexenyl group as described in United States Patent Application Publication No. 20060035254, Manoharan et al., the disclosure of which is incorporated herein in its entirety. Alternatively, or in addition, the oligonucleotide sequence may be conjugated to one or more suitable chemical moieties at one or both ends. For example, the oligonucleotide may be conjugated to cholesterol via a suitable linkage such as a hydroxyprolinol linkage at the 3′ end.

Another aspect of the invention provides an animal model that mimics aspects of the dysregulation of miRNA expression and biogenesis identified in the molecular neuropathology of schizophrenia. For example, transgenic rodents are contemplated which constitutively or inducibly over express one or more of the miRNAs which have been shown to be upregulated in schizophrenia. Alternatively, these miRNAs can be expressed in the brain tissue of adult rodents via transgenes delivered by viral vectors. Synthetic miRNA precursor hairpins or double stranded mature miRNA can also be delivered directly to emulate the conditions observed in schizophrenia. Further, one or more miRNAs could be expressed from a single polycistronic miRNA vector (Liu et al, Nucleic Acids Res 9:2811-2824, 2008). In yet another aspect, DGCR8 or other genes that regulate the biogenesis of miRNA could be introduced into animals at various stages of development to emulate the elevation of cortical miRNA biogenesis observed in schizophrenia. These models provide a new model for schizophrenia for use in a range of applications including drug development or drug screening. In addition, the animal models provide a system for preclinical development and testing of miRNA targeting or miRNA biogenesis targeting medicaments.

The present invention is further described by the following non-limiting Examples.

In the Examples, the materials and methods described below were employed:

### Tissue Collection

Fresh frozen postmortem STG gray matter tissue from 21 subjects with schizophrenia and 21 non-psychiatric controls and DLPFC gray matter from two cohorts of 15 and 36 subjects, respectively, with schizophrenia and non-psychiatric controls was obtained through the NSW Tissue Resource Centre, The University of Sydney, Australia. The grey matter tissue was taken from the outer edge of blocks of STG tissue from the most caudal coronal brain slice containing the STG (Brodmann's Area 22) or DLPFC (Brodmann's Area 9). In all cases, a diagnosis of schizophrenia in accordance with DSM-IV criteria was confirmed by medical file review using the Item Group Checklist of the Schedules for Clinical Assessment in Neuropsychiatry and the Diagnostic Instrument for Brain Studies. Subjects with a significant history of drug or alcohol abuse, or other condition or gross neuropathology that might could influence agonal state were excluded. In addition, control subjects were excluded if there was a history of alcoholism or suicide. All subjects were of Caucasian descent. Subjects with schizophrenia were matched for gender, age, brain hemisphere, PMI and pH.

### Tissue Dissection and RNA Extraction

Postmortem cortical grey matter was dissected from the outer edge of frozen coronal sections (1 cm). In each case approximately 50-60 mg grey matter was removed and immediately homogenized in 1 mL of Trizol reagent and the total RNA extracted according to the manufacturer's instructions (Invitrogen). The RNA concentration and integrity was determined using an Experion bioanalyzer (BioRad).

miRNA Expression Arrays

miRNAs were labeled directly using a ligation approach consisting of 3 μg of total RNA, in 50 mM HEPES pH 7.8, 3.5 mM DTT, 20 mM MgCl₂, 0.1 mM ATP, 10 gg/ml BSA, 10% DMSO, 500 ng 5′-phosphate-cytidyl-uridyl-Cy3-3′ (Dharmacon) and 20 units T4 RNA ligase (Fermentas) (Igloi Anal Biochem 233:124-129, 1996). After incubating for two hours on ice the labeled RNA was precipitated with 0.3 M sodium acetate, 2 volumes 100% v/v ethanol and 20 μg glycogen at −20° C. overnight. A synthetic reference library consisting of oligonucleotides (representing the entirety of miRBase version 7.1) was labeled with Ulysis platinum conjugated AlexaFluor 647 (equivalent to Cy5) for detection in the control channel, using the labeling kit, according to the manufacturer's instructions (Invitrogen). Unconjugated label was then removed by gel filtration through a Sephadex G-25 spin column (GE Healthcare). The labeled reference library was used at a 1/700 dilution, along side the Cy3 labeled miRNAs, in each array hybridization.

Microarrays were prepared using anti-sense LNA oligonucleotides (Exiqon) corresponding to the miRBase Version 7.1 containing 322 human miRNAs sequences (see Supplementary Material). The oligonucleotide probes were printed in duplicate onto GAPS-2 glass slides (Corning). The slides were then prepared and hybridized with the labeled miRNA and synthetic controls as previously described (Thomson et al, Nat methods 1:47-53, 2004). Briefly, slides were pre-hybridized in 3×SSC, 0.1% w/v SDS and 0.2% w/v BSA for 1 hour at 65° C. and washed 4 times with RNAse-free water, once with 100% v/v ethanol, and dried by centrifugation at 150 g for 5 min. Hybridization chambers were, created around each array using 17 mm×28 mm disposable frame seals and cover slides (Bio-Rad). The labeled RNA sample was added to 100 μL hybridization buffer (400 mM Na₂HPO₄ pH 7.0, 0.8% w/v BSA, 5% w/v SDS, 12% v/v formamide) and heated for 4 min at 95° C. (in the dark). The mixture was injected into the chamber and hybridized for 2 hours at 55° C. in a rotary hybridization oven. The coverslips and frames were removed and the slides washed once in 2×SSC, 0.025% w/v SDS at room temperature, 3 times in 0.8×SSC at room temperature and 3 times in ice cold 0.4×SSC. Each slide was then dried by centrifugation for 10 min at 60×g. Arrays were then scanned with a Genepix 4000B Scanner (Axon Instruments) and raw pixel intensities extracted with Genepix Pro 3.0 software (Axon Instruments).

A miRNA was considered expressed if its raw Cy3 pixel intensity was at least 200% above background. Raw Cy3 median pixel intensity values were background subtracted and normalized by U6 snRNA expression. Differential miRNA expression was analyzed using Significance Analysis of Microarrays (SAM) version 2.23 (Stanford University) (Tusher et al, Proc Natl Acad Sci USA 98:5116-5121, 2001) (available on the world-wide-web at -stat.stanford.edu/˜tibs/SAM/. The threshold for significance was set at 5% and a two-class comparison was performed using 5000 permutations of the data. A list of significantly altered miRNAs was compiled (false-discovery rate (FDR) <5%).

### Total RNA Analysis

Total RNA from the STG was quantified using a RNA Quant-it assay according to the manufacturer's instructions (Invitrogen). Equal amounts of individual samples were then pooled for the schizophrenia and control groups respectively. Pooled samples (30 μg) were then dephosphorylated in 1×SAP buffer and 1 unit of shrimp alkaline phosphatase (Fermentas) at 37° C. for 30 min. After heat inactivation, the dephosphorylated RNA was then re-phosphorylated in the presence of [³²P-y] ATP in 1× polynucleotide kinase forward reaction buffer and 1 unit of polynucleotide kinase (Fermentas). Labeled RNA was then combined with an equal volume of formamide/bromophenol blue/25 mM EDTA loading dye and denatured at 95° C. before electrophoresis on a 16% w/v denaturing (TBE/Urea) sequencing gel. The image was generated and analysed from the radiolabeled gel using a Typhoon phosphorimager and ImageQuant software (GE Bioscience).

### Quantitative Real-Time RT-PCR (Q-PCR)

Multiplex reverse transcription was performed on 500 ng of DNaseI treated total RNA using either random hexamers (mRNA analysis), or a combination of reverse primers (miRNA analysis) specific for mature miRNAs, the U6 snRNA, U44 and U49 snoRNAs to a final concentration of 40 nM each. Reactions were performed using Superscript II reverse transcriptase in 1× first strand buffer according to the manufacturer's instructions (Invitrogen). Real-time PCR was performed essentially as previously described (Beveridge et al, Hum Mol Genet 17:1156-1168, 2008) and adapted from Raymond et al, RNA 11.1737-1744, 2005, in triplicate on diluted cDNA combined with Power SybrGreen master mix (Applied Biosystems) with 1 μM of the appropriate forward and reverse primers (Table 2), in a final volume of 12.5 μL using a 7500 Real Time PCR System (Applied Biosystems). Relative miRNA expression was determined by the difference between their individual cycle threshold (Ct) value and that produced in the same sample for the geometric mean of U6, U44 and U49 expression (deltaCt). Similarly, relative mRNA expression ratio was normalized with respect to the geometric mean of GUSB and HMBS expression. Differential expression of a given miRNA or mRNA was determined by the difference between the mean deltaCt for the schizophrenia and control cohorts (deltadeltaCt) expressed as a ratio (2^{-ΔΔCt}) (Livak K J & Schmittgen T D Methods 25:402-408, 2001).

### Statistical Analyses

The distribution of each data set was tested for normality using GraphPad Prism version 4.00. Each data set passes the test for normality and as such, parametric statistical analyses were used. To determine the significance of differential miRNA expression between the two cohorts, an un-paired one-tailed t-test was applied (direction of altered expression was predicted by microarray experiments). Differential gene expression (mRNA) was determined by un-paired two-tailed t-tests. In all cases significance was considered as p<0.05.

### Bioinformatic Analyses

Putative target genes were identified using the publically available database, TargetCombo (which combines information gathered from multiple databases including Diana-microT, PicTar, TargetScanS and miRanda; available on the world-wide web at diana.pcbi.upenn.edu/cgi-bin/TargetCombo.cgi. Pathway analyses of the target gene lists were carried out using the DAVID bioinformatics resource (available on the internet at david.abcc.ncifcrf.gov).

### Cell Culture, Transfection and Target Gene Reporter Assay

HEK-293 cell cultures were maintained as confluent monolayers at 37° C. with 5% v/v CO₂ and 90% v/v humidity in DMEM with 10% v/v foetal calf serum, 20 mM HEPES, 0.15% w/v sodium bicarbonate, and 2 mM L-glutamine. Cells were seeded into 24-well plates and transfected 24 hours later using Lipofectamine 2000 (Invitrogen). In each case transfections were performed according to manufacturer's instructions with 100 nM synthetic miRNA or anti-miR oligonucleotide (see Table 3). Validation of predicted target genes was accomplished by co-transfecting HEK293 cells with synthetic miRNA or an LNA-modified antisense inhibitor and recombinant firefly luciferase reporter gene constructs containing 3′ UTR sequences substituted from the target gene. Oligonucleotides encoding target gene miRNA recognition elements were annealed to form Spel and Hindlll restricted overhangs of a ligatable cassette compatible with Spel and Hindlll digested pMIR-REPORT vector (Ambion) [see Table 3]. Reporter gene silencing in response to miRNA co-transfection was monitored with respect to a control plasmid expressing renilla luciferase (pRL-T\dot{K}) using the dual luciferase reporter assay (Promega).

To control for non-specific effects associated with siRNA transfection, the controls were co-transfected with mutant miRNAs or anti-miRs.

TABLE 3
Oligonucleotide Sequences

| | | | | |
|---|---|---|---|---|
| | | | | SEQ ID |
| Type | Name | Sequence | Target | NO: |

| | | | | |
|---|---|---|---|---|
| Primers^{a} | U6-probe | GCCATGCTAATCTTCTCTGTATC | U6 snRNA | 8 |
| | U6-F339 | CGGCAGCACATATACTAAAATTGG | U6 snRNA | 9 |
| | U49-F | ATCACTAATAGGAAGTGCCGTC | U49 snoRNA | 10 |
| | U49-R | ACAGGAGTAGTCTTCGTCAGT | U49 snoRNA | 11 |
| | U44-F | TGATAGCAAATGCTGACTGA | U44 snoRNA | 12 |
| | U44-R | CAGTTAGAGCTAATTAAGACCT | U44 snoRNA | 13 |
| | 107-F | AGCAGCAYTTGTACAG | miR-107 | 14 |
| | 107-R | GTAAAACGACGGCCAGTTGATAGCC | miR-107 | 15 |
| | 15a-F^{b} | T+AG+CAGCACATAA | miR-15a | 16 |
| | 15a-R | GTAAAACGACGGCCAGTCACAAACCA | miR-15a | 17 |
| | 15b-F | TAGCAGCACATCAT | miR-15b | 18 |
| | 15b-R | GTAAAACGACGGCCAGTTGTAAACC | miR-15b | 19 |
| | 16-F | TAGCAGCACATCAT | miR-16 | 20 |
| | 16-R | GTAAAACGACGGCCAGTTGTAAACC | miR-16 | 21 |
| | 128a-F | TCACAGTGAACCG | miR-128a | 22 |
| | 128a-R | GTAAAACGACGGCCAGTAAAAGAGAC | miR-128a | 23 |
| | 181a-F | AACATTCAACGCTG | miR-181a | 24 |
| | 181a-R | GTAAAACGACGGCCAGTACTCACCGA | miR-181a | 25 |
| | 181b-F | TTTCTAACATTCATTGCT | miR-181b | 26 |
| | 181b-R | CAACCTTCTCCCACCGAC | miR-181b | 27 |
| | 195-F | T+AGCAGCACAGA | miR-195 | 28 |
| | 195-R | GTAAAACGACGGCCAGTGCCAATATT | miR-195 | 29 |
| | 19a-F | TGTGCAAATCTATGC | miR-19a | 30 |
| | 19a-R | GTAAAACGACGGCCAGTTCAGTTTT | miR-19a | 31 |
| | 20a-F | T+AA+AGTGCTTATAGTG | miR-20a | 32 |
| | 20a-R | GTAAAACGACGGCCAGTCTACCTG | miR-20a | 33 |
| | 219-F | T+GAT+TGTCCAAAC | miR-219 | 34 |
| | 219-R | GTAAAACGACGGCCAGTAGAATTGC | miR-219 | 35 |
| | 26b-F | TT+CA+AGTAATTCAGG | miR-26b | 36 |
| | 26b-R | GTAAAACGACGGCCAGTAACCTAT | miR-26b | 37 |
| | 27a-F | TT+CACAGTGGCTA | miR-27a | 38 |
| | 27a-R | GTAAAACGACGGCCAGTGCGGAACT | miR-27a | 39 |
| | 29b-F | T+AG+CACCATTTGAA | miR-29c | 40 |
| | 29c-R | GTAAAACGACGGCCAGTTAACCGAT | miR-29c | 41 |
| | 338-F | AA+CAATATCCTGGT | miR-338 | 42 |
| | 338-R | GTAAAACGACGGCCAGTCACTCAGC | miR-338 | 43 |
| | 7-F | T+GGAAGACTAGTGA | miR-7 | 44 |
| | 7-R | GTAAAACGACGGCCAGTACAACAAAA | miR-7 | 45 |
| | let-7d-F | AGA+GGTAGTAGGTT | let-7d | 46 |
| | let-7d-R | GTAAAACGACGGCCAGTAACTATGC | let-7d | 47 |
| | let-7e-F | TG+AGGTAGGAGGT | let-7e | 48 |
| | let-7e-R | GTAAAACGACGGCCAGTACTATACA | let-7e | 49 |
| | M13-F | GTAAAACGACGGCCAGT | Rev primer | 50 |
| | | | for miRNA | |
| | | | Q-PCR | |
| | GUSB-F | GCCAATGAAACCAGGTATCCC | GUSB | 51 |
| | GUSB-R | GCTCAAGTAAACAGGCTGTTTTCC | GUSB | 52 |
| | HMBS-F | GAGAGTGATTCGCGTGGGTA | HMBS | 53 |
| | HMBS-R | CAGGGTACGAGGCTTTCAAT | HMBS | 54 |
| | FXR2-F | ACCGCCAGCCAGTGACTGTG | FXR2 | 55 |
| | FXR2-R | AGTCACCCTTCTGTCCTGAAA | FXR2 | 56 |
| | DICER1-F | CACATCAATAGATACTGTGCT | DICER | 57 |
| | DICER-R | TTGGTGGACCAACAATGGAGG | DICER | 58 |
| | DGCR8-F | GCTGAGGAAAGGGAGGAG | DGCR8 | 59 |
| | DGCR8-R | ACGTCCACGGTGCACAG | DGCR8 | 60 |
| | DROSHA-F | AAGCGTTAATAGGAGCTGTTTACT | DROSHA | 61 |
| | DROSHA-R | CGTCCAAATAACTGCTTGGCT | DROSHA | 62 |
| | XPO5-F | ATATATGAGGCACTGCGCC | EXP-5 | 63 |
| | XPO5-R | AAACTGGTCCAGTGAGTCCTT | EXP-5 | 64 |
| | DDX26-F2 | AGATCCGAAAGCCAGGAAGAAAA | DDX26 | 65 |
| | DDX26-R2 | TTTGTAAACTGCCTTGCACATGC | DDX26 | 66 |
| | DDX5-F | AAGGATGAAAAACTTATTCGT | DDX5 | 67 |
| | DDX5-R | TTTTCCATGTTTGAATTCATT | DDX5 | 68 |
| | DDX17-F | GTGAAAAAGACCACAAGTTGA | DDX17 | 69 |
| | DDX17-R | TACACATAGCTGGCCAACCAT | DDX17 | 70 |
| | FXR2-F | ACCGCCAGCCAGTGACTGTG | FXR2 | 71 |
| | FXR2-R | AGTCACCCTTCTGTCCTGAAA | FXR2 | 72 |
| | pri-181b-2-F1 | AAGAAGAGCCAGGAGTCAGC | pri-181b-2 | 73 |
| | pri-181b-2-R1 | TCAGTTGGTGGGGTrGCCTT | pri-181b-2 | 74 |
| | pre-181b-2-F | CTGATGGCTGCACTCAACAT | pre-181b | 75 |
| | pre-181b-2-R | TGATCAGTGAGTTGATTCAGACT | pre-181b | 76 |
| | pri-26b-F | CCGTGCTGTGCTCCCT | pri-26b | 77 |
| | pri-26b-R | CGAGCCAAGTAATGGAGAACAG | pri-26b | 78 |
| | pre-26b-F | GACCCAGTTCAAGTAATTCAGGA | pre-26b | 79 |
| | pre-26b-R | CGAGCCAAGTAATGGAGAACAG | pre-26b | 80 |
| Cassettes^{c} | VSNL1-107-T | CTAGTTCCTCCAAAGCCTGGGCAGAAATGTGCT | VSNL1 | 81 |
| | | GCAAA | | |
| | VSNL1-107-B | AGCTTTTGCAGCACATTTCTGCCCAGGCTTTGG | VSNL1 | 82 |
| | | AGGAA | | |
| | RELN-107-T | CTAGTTTACTTGTTATGTTGTAATATTTTGCTGC | RELN | 83 |
| | | TGAATTT | | |
| | RELN-107-B | AGCTAAATTCAGCAGCAAAATATTACAACATAA | RELN | 84 |
| | | CAAGTAAA | | |
| | HTR2A-107-T | CTAGCTATTTTCAAGTGGAAACCTTGCTGCTAT | HTR2A | 85 |
| | | GCTGTTCA | | |
| | HTR2A-107-B | AGCTTGAACAGCATAGCAGCAAGGTTTCCACTT | HTR2A | 86 |
| | | GAAAATAG | | |
| | GRIN3A-107-T | CTAGGCACAAACCCTATCAAGAGCTGCTGCTTC | GRIN3A | 87 |
| | | CCT | | |
| | GRIN3A-107-B | AGCTAGGGAAGCAGCAGCTCTTGATAGGGTTTG | GRIN3A | 88 |
| | | TGC | | |
| | PLEXNA2-107-T | CTAGGACAGTTCTGCCTCTGTGACTGCTGCTTT | PLEXNA2 | 89 |
| | | GCATG | | |
| | PLEXNA2-107-B | AGCTCATGCAAAGCAGCAGTCACAGAGGCAGA | PLEXNA2 | 90 |
| | | ACTGTC | | |
| | DLG4-107-T | CTAGGTCCGGGAGCCAGGGAAGACTGGAAATG | DLG4 | 91 |
| | | CTGCCG | | |
| | DLG4-107-B | AGCTCGGCAGCATTTCCAGTCTTCCCTGGCTCC | DLG4 | 92 |
| | | CGGAC | | |
| | DRD1-107-T | CTAGAATTTACGATCTTAGGTGGTAATGAAAAG | DRD1 | 93 |
| | | TATATGCTGCTTTG | | |
| | DRD1-107-B | AGCTCAAAGCAGCATATACTTTTCATTACCACC | DRD1 | 94 |
| | | TAAGATCGTAAATT | | |
| | GRM7-107-T | CTAGGTTTGTAATAAGTACTTTCGTTAATCTTGC | GRM7 | 95 |
| | | TGCTTATGTG | | |
| | GRM7-107-B | AGCTCACATAAGCAGCAAGATTAACGAAAGTA | GRM7 | 96 |
| | | CTTATTACAAAC | | |
| | RGS4-107-T | AATGCACTAGTCCACATTGTAGCCTAATATTCA | RGS4 | 97 |
| | | TGCTGCCTGCCATGAAGCTTAATGC | | |
| | RGS4-107-B | GCATTAAGCTTCATGGCAGGCAGCATGAATATT | RGS4 | 98 |
| | | AGGCTACAATGTGGACTAGTGCATT | | |
| miRNA^{d} | miR-107+ | AGCAGCAUUGUACAGGGCUAUCA | miR-107 | 99 |
| | miR-107− | AUAGCCCUGUACAAUGCUGUAUU | miR-107 | 100 |
| | miR-15a+ | UAGCAGCACAUAAUGGUUUGUG | miR-15a | 101 |
| | miR-15a− | CAAACCAUUAUGUGCUGUUAUU | miR-15a | 102 |
| | miR-15b+ | UAGCAGCACAUCAUGGUUUACA | miR-15b | 103 |
| | miR-15b− | UAAACCAUGAUGUGCUGUUAUU | miR-15b | 104 |
| | miR-16+ | UAGCAGCACGUAAAUAUUGGCG | miR-16 | 105 |
| | miR-16− | CCAAUAUUUACGUGCUGUUAUU | miR-16 | 106 |
| | miR-195+ | UAGCAGCACAGAAAUAUUGGC | miR-195 | 107 |
| | miR-195− | CAAUAUUUCUGUGCUGUUAUU | miR-195 | 108 |
| | control-miRNA-1+ | AUCCACCACGUAAAUAUUGGCG | miR-15 family | 109 |
| | control-miRNA-1− | CCAAUAUUUACGUGGUGGAUCG | miR-15 family | 110 |
| | control-miRNA-2+ | UCCACCAAUGUACAGGGCUAUCA | miR-107 | 111 |
| | control-miRNA-2− | AUAGCCCUGUACAUUGGUGAAUU | miR-107 | 112 |
| Anti-miR^{e} | anti-miR-107 | T+GAT+AGC+CCT+GTA+CAA+TGC+TG | miR-107 | 113 |
| | anti-miR-15a | C+ACA+AAC+CAT+TAT+GTG+CTG+CTA | miR-15a | 114 |
| | anti-miR-15b | T+GTA+AAC+CAT+GAT+GTG+CTG+CTA | miR-15b | 115 |
| | anti-miR-16 | C+GCC+AAT+ATT+TAC+GTG+CTG+CTA | miR-16 | 116 |
| | anti-miR-195 | G+CCA+ATA+TTT+CTG+TGC+TGC+TA | miR-195 | 117 |
| | control anti-miR-1 | C+GCC+AAT+ATT+TAC+GTG+GTG+GAT | miR-15 family | 118 |
| | control anti-miR-2 | T+GAT+AGC+CCT+GTA+CAT+TGG+TG | miR-107 | 119 |

^{a}The direction of primers with respect to the target sequence was denoted in the name as either F or R for forward and reverse respectively. Underlined sequence is not gene specific and was used to provide a primer recognition sequence. For miRNA Q-PCR, the Rev primer is used for reverse transcription, and the For primer is used in the Q-PCR with M13-F as the reverse primer ^{b}The positions of LNA modified bases are preceded by a “+” symbol. ^{c}SpeI/HindIII cassettes containing putative target recognition elements were used to generate recombinant luciferase reporter gene constructs. “T” indicates top strand and “B” indicates bottom strand. ^{d}Synthetic miRNA are used to over-express microRNA. “+” indicates top strand and “−” indicates bottom strand. ^{e}Anti-miRs are used to suppress endogenous microRNA.

### Example 1

### Elevation of miRNA Expression in the STG in Schizophrenia

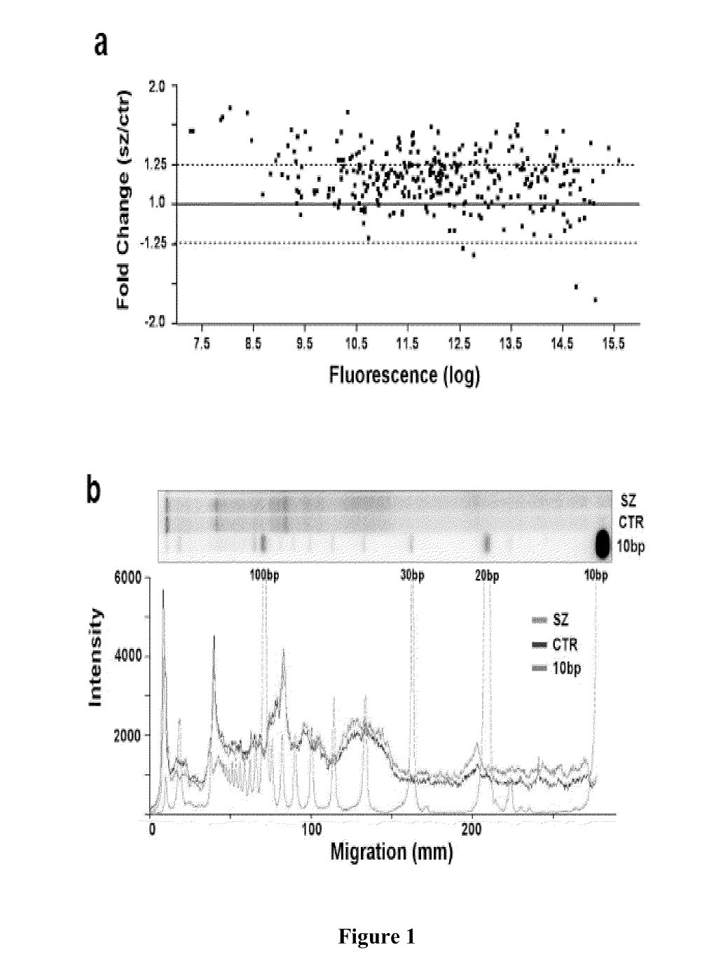

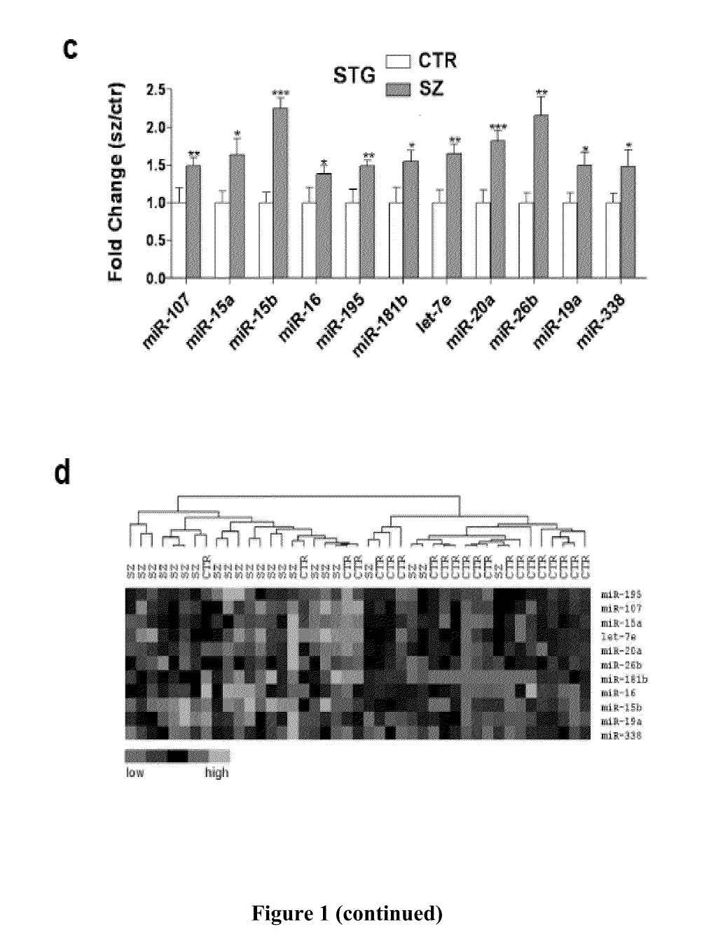

Changes in miRNA expression have broad implications for disease, as each miRNA molecule is capable of influencing the expression of hundreds of target genes. The expression of miRNA has been shown to be important during development, particularly in the mammalian brain (Sempere et al, Genome Biol 5:R13, 2004), so it is plausible that these molecules have great significance in neurodevelopmental disorders such as schizophrenia. In this study miRNA expression in the STG was investigated (Brodmann's Area 22, 17 matched pairs of schizophrenia and nonpsychiatric controls) and the DLPFC (Brodmann's Area 9, 15 and 37 matched pairs), using a microarray printed with LNA modified capture probes corresponding to miRBase version 7.1 (Exiqon) (Thomson et al, 2004 supra). The arrays were also furnished with two probes specific for different sites in the U6 small nuclear RNA (snRNA) that enabled external or miRNA-independent normalization of miRNA expression between samples. In this analysis, miR-181b (previously found to be up-regulated in the STG) represented only one of many significantly elevated miRNAs in the schizophrenia group. This observation, apparent in scatter plots of the average expression between schizophrenia and controls for each miRNA (FIG. 1A), implied there was a schizophrenia-associated global elevation of miRNA expression in the STG. The significance of these changes was supported by Significance Analysis of Microarrays (SAM), which reported that 59 miRNAs (or 21% of expressed miRNA) were up-regulated (false discovery rate <5%; two-class analysis; 5000 permutations of the data; p<0.05 Student's t-test). With the apparent scope of this alteration in small RNA expression, consideration was given that it might be possible to directly visualize this in the small RNA fraction of (Igloi G L 1996 supra) ³²P-labeled total RNA separated on a sequencing gel. This experiment revealed that the 22 nt band (corresponding with miRNAs) was at least 1.5 times more intense for the schizophrenia sample than that of the controls; and was indicative of a general increase in small RNA expression in schizophrenia (FIG. 1b).

For more specific evidence of this phenomenon, quantitative real-time RT-PCR (Q-PCR) assays for eleven miRNA shown to be among the most significantly up regulated by microarray analysis were established. The relative expression values for each miRNA across an extended cohort, consisting of 21 samples, and 21 matched controls of postmortem cortical grey matter from the STG, were normalized with respect to the geometric mean of three constitutively expressed small RNAs (including U6 snRNA, U44 and U49 snoRNA) [FIG. 1c]. In each case the level of concordance with the microarray and Q-PCR was very high and in many cases the average schizophrenia-associated increase was even greater by Q-PCR than that observed by microarray. This trend was also highly visible in individual samples clustered by expression and visualized by heat map (FIG. 1d). Hierarchical clustering analysis of these differentially expressed miRNA was characterized by a high degree of segregation between the schizophrenia and control groups. Prominent among this group of miRNA associated with schizophrenia was the apparent up-regulation of the entire miR-15 family; consisting of miR-15a, miR-15b, miR-16 and miR-195, which all share the same functionally important seed pairing region and consequently a large proportion of target genes. In addition, miR-107 was among the most significantly up-regulated in the STG and also shares a high degree of seed region homology with the miR15 family (FIG. 1c). Collectively they are predicted to target a wide array of target genes, many implicated in schizophrenia including; brain BDNF, NRG1, RELN, DRD1, HTR4, GABR1, GRIN, GRM7, CHRM1 and ATXN2.

### Example 2

### Elevation of miRNA Expression in the DLPFC in Schizophrenia

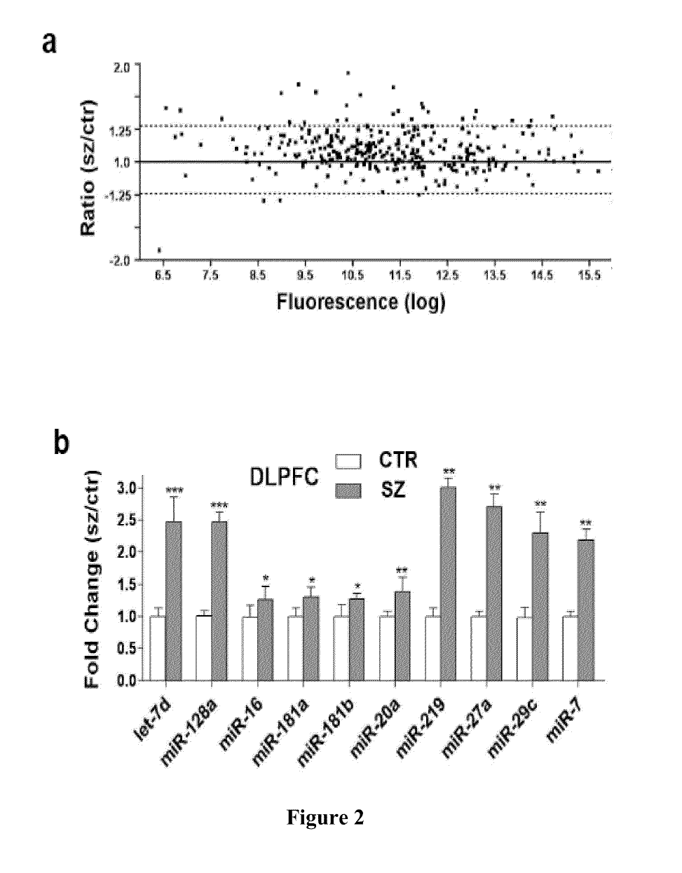

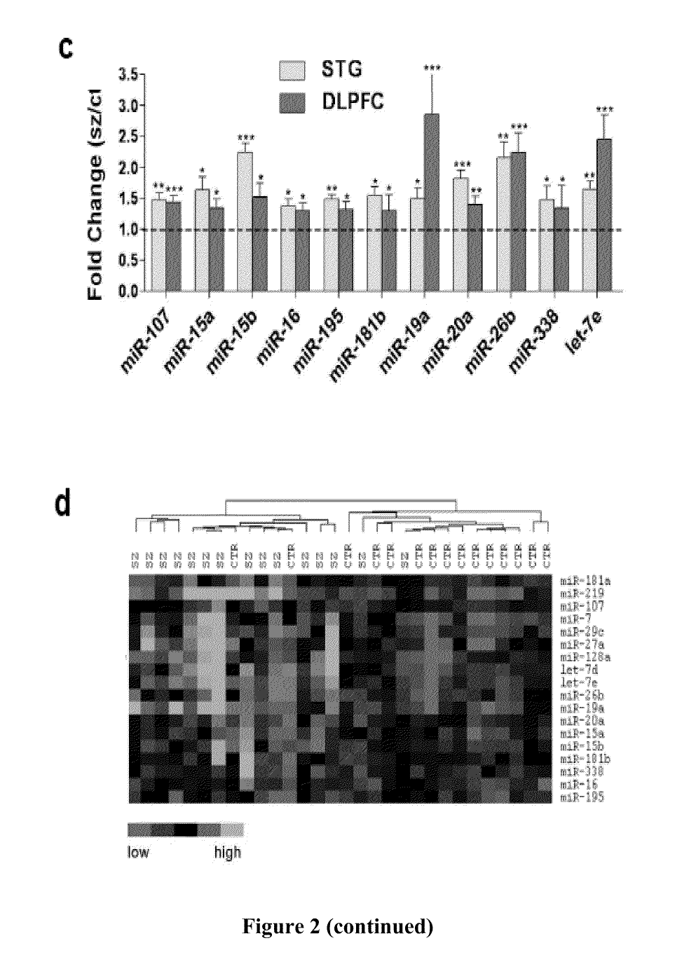

In view of the possibility that these changes in miRNA expression were merely STG related phenomena, similar investigations were initiated of the DLPFC (BA9); a region most frequently identified in the neuropathology of schizophrenia. Total RNA from postmortem grey matter from two cohorts of 15 and 37 cases, respectively, with a history of schizophrenia and matched controls with no record of psychiatric illness, was extracted and subjected to microarray analysis as described for the STG. The miRNA expression profile in this tissue was similar to that in the STG, with 274 expressed miRNAs (compared to 280 in the STG). Importantly, the DLPFC demonstrated a schizophrenia-associated global increase in miRNA expression that was broadly consistent with the observations in the STG (FIG. 2A). According to SAM analysis, 26 (9.5% of expressed miRNA) were significantly up-regulated including miR-181b, miR-15, miR-20a, miR-184 and miR-338, and these were also significantly increased in the STG.

Again, to validate the microarray results, Q-PCR assays were performed on a subset of ten differentially expressed miRNA as described for the STG using the expression of three constitutively expressed small RNAs as a reference. This analysis supported the array findings and in some cases exceeded expectation by showing an even stronger schizophrenia-associated up-regulation in miRNA (FIG. 2b). Four of the tested miRNAs including miR-181b, miR-16, miR-20a and miR-338 were also up-regulated in the STG cohort (FIG. 1d). To determine if the differentially expressed miRNA in common extended beyond the scope identified by SAM analysis of the DLPFC microarray experiment, the DLPFC samples were also examined for the remaining schizophrenia-associated miRNAs validated for the STG cohort. These miRNA, including let-7e, miR-19a, miR-26b, miR-338, miR-107 and the remaining members of the miR-15 family, were all found to be significantly up-regulated in the DLPFC as well as the STG by Q-PCR (FIG. 2c). miR-181c and miR-328 were also shown to be upregulated in the DLPFC from schizophrenics. In a similar manner to the STG cohort, unsupervised hierarchical clustering of miRNA expression in individual DLPFC samples also produced very good segregation between the schizophrenia and control groups (FIG. 2d). A summary of expressed miRNAs is provided in Table 4.

TABLE 4

| | | Relative | Microarray | | | |
|---|---|---|---|---|---|---|
| | | Expression | fold- | | | RT-PCR |
| | miRNA | Value | change | FDR (%) | p-value | validation |
| STG | | | | | | |
| 1 | hsa-let-7e | 35988 | 1.42 | 0 | <0.001 | * |
| 2 | hsa-miR-107 | 7563 | 1.42 | 0 | 0.018 | * |
| 3 | hsa-miR-125b | 13635 | 1.29 | 3.84 | 0.168 | |
| 4 | hsa-miR-128a | 12396 | 1.32 | 0 | 0.140 | |
| 5 | hsa-miR-128b | 12670 | 1.44 | 0 | 0.110 | |
| 6 | hsa-miR-129 | 5657 | 1.48 | 0 | 0.088 | |
| 7 | hsa-miR-130a | 2555 | 1.44 | 0 | 0.037 | |
| 8 | hsa-miR-133b | 2145 | 1.43 | 0 | 0.064 | |
| 9 | hsa-miR-138 | 9339 | 1.56 | 0 | 0.003 | |
| 10 | hsa-miR-146b | 1683 | 1.51 | 0 | 0.062 | |
| 11 | hsa-miR-148a | 824 | 1.56 | 0 | 0.002 | |
| 12 | hsa-miR-150 | 2912 | 1.31 | 0 | 0.025 | |
| 13 | hsa-miR-152 | 858 | 1.60 | 0 | 0.011 | |
| 14 | hsa-miR-155 | 3606 | 1.52 | 0 | 0.096 | |
| 15 | hsa-miR-15a | 4619 | 1.39 | 0 | 0.044 | * |
| 16 | hsa-miR-15b | 3917 | 1.53 | 0 | 0.007 | * |
| 17 | hsa-miR-16 | 8209 | 1.54 | 0 | 0.136 | * |
| 18 | hsa-miR-17-3p | 1388 | 1.64 | 0 | 0.053 | |
| 19 | hsa-miR-17-5p | 4812 | 1.33 | 0 | 0.136 | |
| 20 | hsa-miR-195 | 4750 | 1.42 | 0 | 0.008 | * |
| 21 | hsa-miR-197 | 10552 | 1.24 | 0 | 0.109 | |
| 22 | hsa-miR-199a* | 1100 | 1.59 | 0 | 0.061 | |
| 23 | hsa-miR-19a | 963 | 1.65 | 0 | 0.018 | * |
| 24 | hsa-miR-20a | 9012 | 1.71 | 0 | 0.030 | * |
| 25 | hsa-miR-222 | 5853 | 1.35 | 0 | 0.109 | |
| 26 | hsa-miR-23a | 12987 | 1.51 | 0 | 0.102 | |
| 27 | hsa-miR-24 | 887 | 1.54 | 0 | 0.050 | |
| 28 | hsa-miR-26b | 19121 | 1.62 | 0 | 0.006 | * |
| 29 | hsa-miR-27b | 8698 | 1.28 | 0 | 0.196 | |
| 30 | hsa-miR-28 | 3596 | 1.47 | 0 | 0.107 | |
| 31 | hsa-miR-296 | 3499 | 1.58 | 0 | 0.048 | |
| 32 | hsa-miR-328 | 5943 | 1.30 | 3.84 | 0.239 | |
| 33 | hsa-miR-330 | 1968 | 1.55 | 0 | 0.098 | |
| 34 | hsa-miR-335 | 11056 | 1.54 | 0 | 0.033 | |
| 35 | hsa-miR-338 | 25545 | 1.55 | 0 | 0.238 | * |
| 36 | hsa-miR-339 | 1254 | 1.63 | 0 | 0.054 | |
| 37 | hsa-miR-340 | 1159 | 1.48 | 0 | 0.043 | |
| 38 | hsa-miR-373* | 14328 | 1.31 | 0 | 0.144 | |
| 39 | hsa-miR-381 | 1420 | 1.61 | 0 | 0.003 | |
| 40 | hsa-miR-409-5p | 2579 | 1.57 | 0 | 0.054 | |
| 41 | hsa-miR-432* | 3530 | 1.34 | 0 | 0.090 | |
| 42 | hsa-miR-452* | 1477 | 1.60 | 0 | 0.074 | |
| 43 | hsa-miR-455 | 556 | 1.78 | 0 | 0.014 | |
| 44 | hsa-miR-484 | 2277 | 1.45 | 0 | 0.021 | |
| 45 | hsa-miR-485-5p | 906 | 1.60 | 0 | 0.012 | |
| 46 | hsa-miR-486 | 5375 | 1.40 | 0 | 0.122 | |
| 47 | hsa-miR-487a | 7270 | 1.52 | 0 | 0.020 | |
| 48 | hsa-miR-489 | 1506 | 1.62 | 0 | 0.006 | |
| 49 | hsa-miR-494 | 26241 | 1.57 | 0 | 0.125 | |
| 50 | hsa-miR-499 | 2086 | 1.49 | 0 | 0.023 | |
| 51 | hsa-miR-502 | 10657 | 1.38 | 0 | 0.212 | |
| 52 | hsa-miR-517a | 3261 | 1.56 | 0 | 0.054 | |
| 53 | hsa-miR-517c | 912 | 1.71 | 0 | 0.008 | |
| 54 | hsa-miR-518b | 2954 | 1.43 | 0 | 0.042 | |
| 55 | hsa-miR-519d | 47530 | 1.41 | 0 | 0.064 | |
| 56 | hsa-miR-520a* | 1164 | 1.56 | 0 | 0.053 | |
| 57 | hsa-miR-520g | 1748 | 1.67 | 0 | 0.361 | |
| 58 | hsa-miR-9* | 7050 | 1.28 | 3.84 | 0.170 | |
| 59 | hsa-miR-99a | 3439 | 1.39 | 0 | 0.053 | |
| DLPFC | | | | | | |
| 1 | hsa-let-7d | 25656 | 1.30 | 0 | 0.019 | * |
| 2 | hsa-miR-101 | 6327 | 1.23 | 0 | 0.071 | |
| 3 | hsa-miR-105 | 2930 | 1.26 | 0 | 0.045 | |
| 4 | hsa-miR-126* | 1303 | 1.61 | 0 | 0.035 | |
| 5 | hsa-miR-128a | 11797 | 1.19 | 0 | 0.033 | * |
| 6 | hsa-miR-153 | 6314 | 1.37 | 0 | 0.091 | |
| 7 | hsa-miR-16 | 3073 | 1.20 | 0 | 0.111 | * |
| 8 | hsa-miR-181a | 3362 | 1.27 | 0 | 0.048 | * |
| 9 | hsa-miR-181b | 3263 | 1.20 | 0 | 0.052 | * |
| 10 | hsa-miR-181d | 3263 | 1.23 | 0 | 0.087 | |
| 11 | hsa-miR-184 | 19899 | 1.18 | 0 | 0.100 | |
| 12 | hsa-miR-199a | 1191 | 1.41 | 0 | 0.086 | |
| 13 | hsa-miR-20a | 2041 | 1.29 | 0 | 0.058 | * |
| 14 | hsa-miR-219 | 6716 | 1.42 | 0 | 0.084 | * |
| 15 | hsa-miR-223 | 3203 | 1.42 | 0 | 0.090 | |
| 16 | hsa-miR-27a | 4104 | 1.27 | 0 | 0.014 | * |
| 17 | hsa-miR-29c | 18919 | 1.23 | 0 | 0.091 | * |
| 18 | hsa-miR-302a* | 950 | 1.28 | 0 | 0.009 | |
| 19 | hsa-miR-302b* | 1591 | 1.21 | 0 | 0.039 | |
| 20 | hsa-miR-31 | 578 | 1.35 | 0 | 0.024 | |
| 21 | hsa-miR-33 | 623 | 1.64 | 0 | 0.009 | |
| 22 | hsa-miR-338 | 12394 | 1.33 | 0 | 0.033 | * |
| 23 | hsa-miR-409-3p | 803 | 1.26 | 0 | 0.035 | |
| 24 | hsa-miR-512-3p | 915 | 1.23 | 0 | 0.035 | |
| 25 | hsa-miR-519b | 3226 | 1.42 | 0 | 0.109 | |
| 26 | hsa-miR-7 | 3112 | 1.47 | 0 | 0.011 | |

P values were derived for unpaired comparisons using a two tailed t-test

### Example 3

### Altered miRNA Biogenesis in the STG and DLPFC

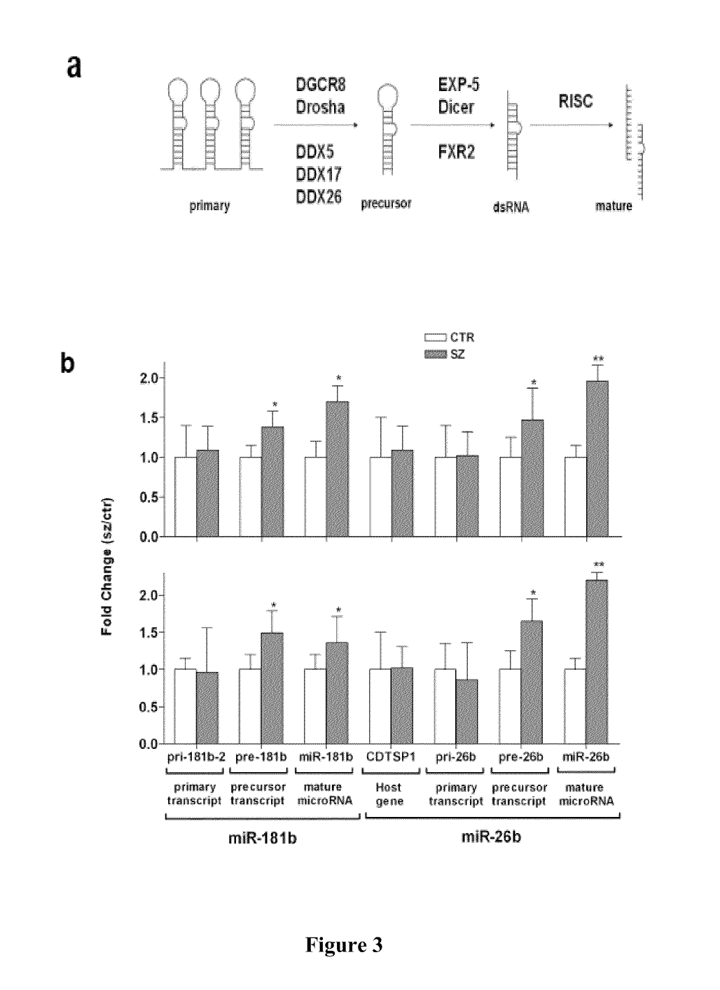

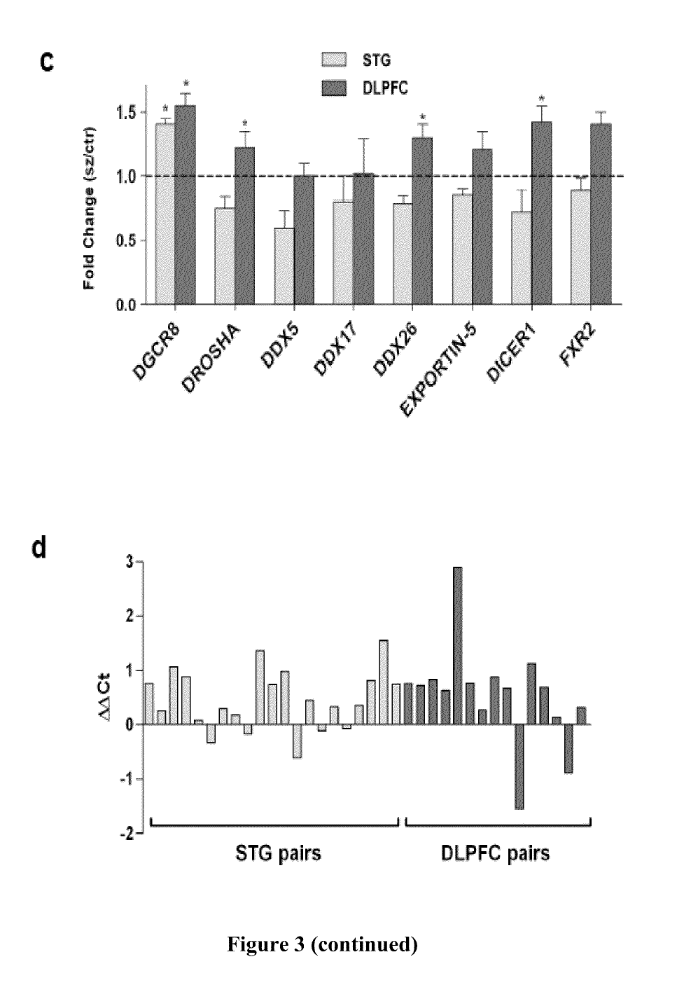

The scope and consistency of the schizophrenia-associated increase in miRNA expression resulted in further investigations on both miRNA processing and the activity of genes in the miRNA biogenesis pathway in this context. For this purpose the relative expression of primary miRNA (pri-miRNA) and precursor miRNA (pre-miRNA) was investigated in addition to the mature miRNA transcripts for miR-181b and miR-26b. Interestingly, while there was a significant increase in pre-miRNA species (consistent with the mature miR-181b and miR-26b), there was no difference in transcription of the pri-miRNA, or the host gene mRNA (CDTSPI) for the intronic miR-26b (FIG. 3b). This supported the hypothesis that there was a schizophrenia-associated increase in miRNA biogenesis rather than any change at the level of miRNA transcription. To further support this assertion, the expression of the microprocessor constituents Drosha and DGCR8 involved in primary miRNA processing were examined (Gregory et al, Nature 432:235-240, 2004). The mRNA for both of these microprocessor components was found to be significantly up-regulated in the DLPFC, and DGCR8 was also up-regulated in the STG (FIG. 3c). Importantly, DGCR8 was shown to be up-regulated in 13 of the 15 matched pairs of DLPFC tissue, and in 16 of the 21 matched pairs of STG (FIG. 3d). These components are thought to be rate limiting in the miRNA biogenesis pathway (Thomson et al, Genes Dev 20:2202-2207, 2006) and as a consequence their elevation in schizophrenia represents a highly plausible explanation for the corresponding increase in both pre-miRNA and mature miRNA expression. The expression of additional genes implicated in primary miRNA processing including the deadbox helicases DDX5 and DDX17, and others with no known association with miRNA processing (e.g. DDX26; differentially expressed in earlier schizophrenia related microarray experiments in STG (Bowden et al, 2008 supra)) were also examined. While both DDX26 was significantly up-regulated in the DLPFC, this trend was not supported in the STG. Its role in miRNA biogenesis, if any, has not been characterized, whereas DDX5 and DDX17 known to be involved in this pathway did not appear significantly altered in either part of the cerebral cortex. The difference in magnitude observed in differential miRNA and pre-miRNA expression (FIG. 3b) was possibly due to some dilution of the pre-miRNA by pri-miRNA template as the pre-miRNA primer set has the capacity to amplify both of these sequences. However, it is also conceivable that other influences downstream of the microprocessor could further elevate mature miRNA expression and contribute to this difference. In this regard the expression of Exportin-5, Dicer and FXR2 by QPCR were examined and found that Dicer was also significantly up-regulated in schizophrenia in the DLPFC (FIG. 3c).

### Example 4

### Biological Consequences of Altered miR-15 Family and miR-107 Expression

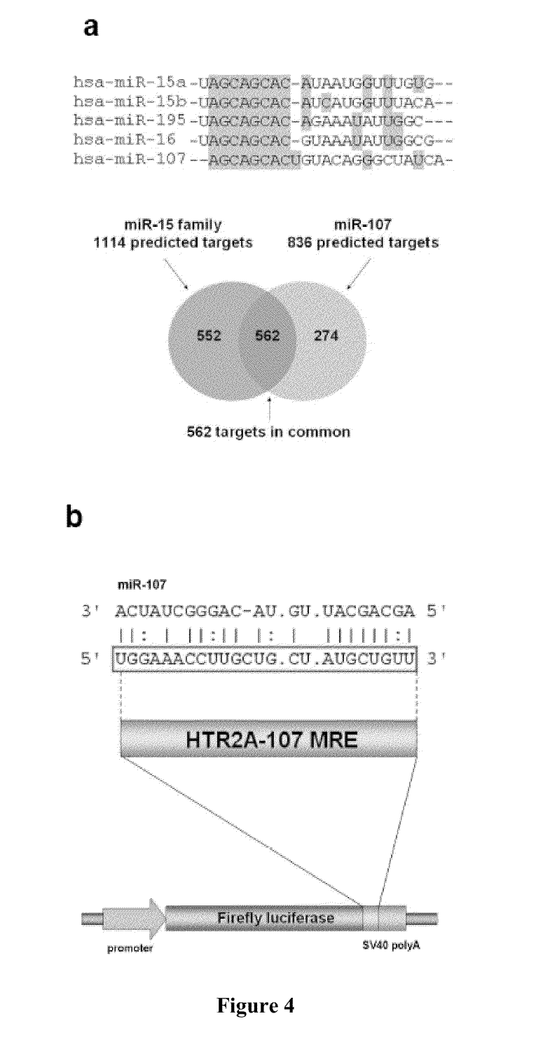

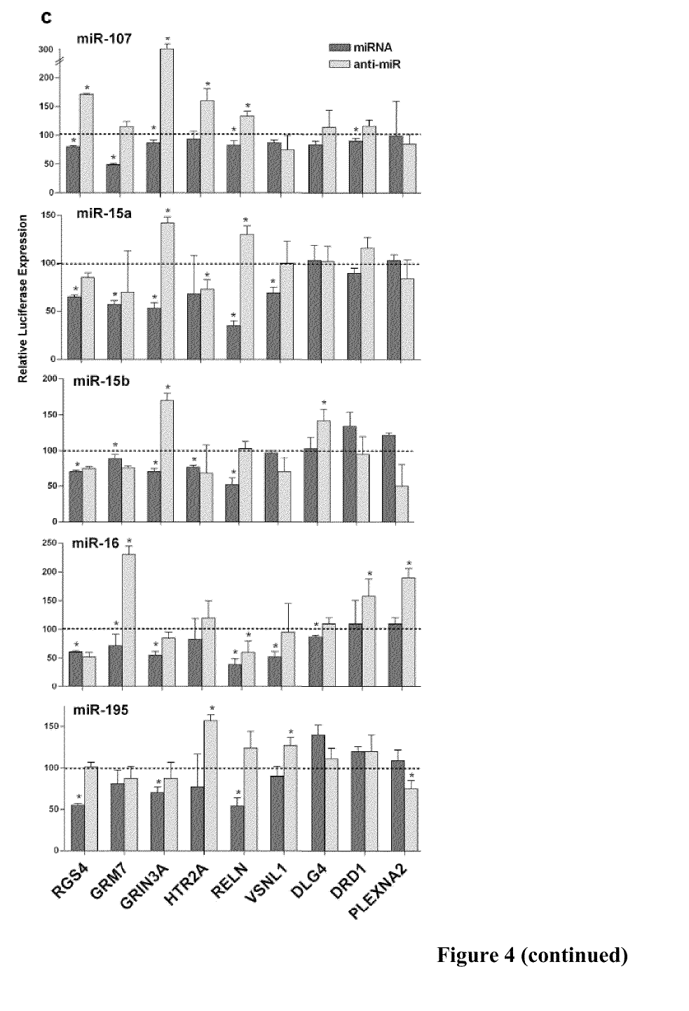

To gain some appreciation of the biological implications of changes in miRNA expression observed in schizophrenia, predicted miRNA targets and their associated pathways were examined to see if any patterns emerged. A conspicuous aspect of miRNA expression analyses in the STG and DLPFC was the prominence of all members of the miR-15 family and miR-107, which all share a similar seed region (FIG. 4a). To ascertain an overall perspective of this influence, a collection of predicted target genes derived using a range of search algorithms (collated on the TargetCombo web service on the world-wide web at diana.pcbi.upenn.edu/cgi-bin/TargetCombo.cgi) (Sethupathy et al, Nat Methods 3:881-886, 2006) was subjected to pathway analysis using the DAVID bioinformatics resource (available on the internet at david.abcc.ncifcrf.gov). (Dennis et al, Genome Biol 4:P3, 2003). Predicted target genes common to the miR-15 family and miR-107, were highly enriched in pathways involved in neural connectivity and synaptic plasticity, including axon guidance, long term potentiation, WNT, ErbB and MAP kinase signaling (Table 5). These processes are repeatedly implicated in the pathophysiology of schizophrenia and a number of individual genes have been shown to be associated with schizophrenia.

TABLE 5

| KEGG Pathway Term | Count | Genes |
|---|---|---|
| Wnt signaling pathway | 17 | TBL1XR1, BTRC, NFATC3, LRP6, WNT7A, CREBBP, APC, PPP2R1A, |
| | | SMAD3, NFATC4, PPP3CB, CAMK2G, WNT3A, FZD10, FZD7, SIAH1, AXIN2 |
| MAPK signaling pathway | 17 | PPM1A, MAP3K4, AKT3, PDGFRB, MAP3K3, RPS6KA3, MAP2K3, |
| | | MAP2K1, FGF2, NFATC4, BDNF, PPP3CB, CDC42, RAF1, CACNB1, CRKL, MRAS |
| Focal adhesion | 16 | ZYX, ITGA9, BCL2, AKT3, PIK3R1, PDGFRB, LAMC1, RELN, MAP2K1, |
| | | CDC42, RAF1, COL1A1, BIRC4, ITGA2, CRKL, VCL |
| Regulation of actin cytoskeleton | 13 | ITGA9, PIK3R1, PDGFRB, APC, MAP2K1, FGF2, CDC42, PPP1R12B, RAF1, |
| | | CRKL, ITGA2, MRAS, VCL |
| Axon guidance | 12 | SEMA3D, PLXNA2, EPHA1, EPHA7, NFATC4, PPP3CB, CDC42, NFATC3, |
| | | SEMA6D, EFNB1, EFNB2, GNAI3 |
| Colorectal cancer | 11 | APC, MAP2K1, BCL2, SMAD3, AKT3, RAF1, PIK3R1, PDGFRB, FZD10, FZD7, AXIN2 |
| Ubiquitin mediated proteolysis | 11 | UBE2A, CUL5, UBE2R2, FBXW7, BTRC, CUL2, SIAH1, BIRC4, UBE2Q1, PIAS1, UBE2J1 |
| Cell cycle | 11 | CDC25A, YWHAG, YWHAQ, CHEK1, SMAD3, CDK6, WEE1, CCNE1, |
| | | E2F3, CREBBP, YWHAH |
| Small cell lung cancer | 10 | BCL2, CDK6, AKT3, CCNE1, PIK3R1, E2F3, BIRC4, ITGA2, PIAS1, LAMC1 |
| Chronic myeloid leukemia | 10 | MAP2K1, SMAD3, CDK6, AKT3, RUNX1, RAF1, PIK3R1, E2F3, CRKL, BCR |
| Prostate cancer | 10 | MAP2K1, BCL2, AKT3, CCNE1, RAF1, PIK3R1, E2F3, PDGFRB, CREB5, CREBBP |
| Melanogenesis | 10 | MAP2K1, RAF1, CAMK2G, WNT3A, GNAO1, FZD10, FZD7, WNT7A, GNAI3, CREBBP |
| Insulin signaling pathway | 10 | RPS6KB1, TRIP10, PPARGC1A, MAP2K1, AKT3, RAF1, FASN, PIK3R1, CRKL, FLOT2 |
| VEGF signaling pathway | 9 | MAP2K1, NFATC4, PPP3CB, CDC42, AKT3, RAF1, PIK3R1, NFATC3, SH2D2A |
| TGF-beta signaling pathway | 9 | ACVR2A, RPS6KB1, PPP2R1A, BMPR1B, CHRD, SMAD3, CREBBP, SMAD5, SMAD7 |
| Acute myeloid leukemia | 8 | RUNX1T1, RPS6KB1, PIM1, MAP2K1, AKT3, RUNX1, RAF1, PIK3R1, |
| ErbB signaling pathway | 8 | RPS6KB1, MAP2K1, AKT3, RAF1, PIK3R1, CAMK2G, CRKL, NRG1 |
| Melanoma | 8 | MAP2K1, FGF2, CDK6, AKT3, RAF1, PIK3R1, E2F3, PDGFRB |
| Glioma | 8 | MAP2K1, CDK6, AKT3, RAF1, PIK3R1, CAMK2G, E2F3, PDGFRB |
| Pancreatic cancer | 8 | MAP2K1, SMAD3, CDK6, CDC42, AKT3, RAF1, PIK3R1, E2F3 |
| Renal cell carcinoma | 8 | MAP2K1, CUL2, CDC42, AKT3, RAF1, PIK3R1, CRKL, CREBBP |
| Non-small cell lung cancer | 7 | MAP2K1, CDK6, AKT3, RAF1, PIK3R1, E2F3, RASSF5, |
| Long-term potentiation | 7 | GRIN1, MAP2K1, PPP3CB, RAF1, CAMK2G, RPS6KA3, CREBBP |
| Basal cell carcinoma | 6 | APC, WNT3A, FZD10, FZD7, WNT7A, AXIN2 |
| Endometrial cancer | 6 | APC, MAP2K1, AKT3, RAF1, PIK3R1, AXIN2 |
| mTOR signaling pathway | 6 | RPS6KB1, CAB39, AKT3, PIK3R1, RPS6KA3, EIF4B |
| p53 signaling pathway | 6 | CHEK1, PPM1D, CDK6, CCNE1, BAI1, SIAH1 |
| Neurodegenerative Diseases | 5 | APP, FBXW7, BCL2, APBA1, CREBBP |
| Circadian rhythm | 3 | CLOCK, BHLHB3, PER1 |

### Example 5

### Validation miRNA Function Using Reporter Gene Assay

To substantiate a link between these schizophrenia-associated target genes and altered expression in this group of miRNA, the respective miRNA recognition elements (MRE) from nine target genes including RGS4, GRM7, GRIN3A, HTR2A, RELN, VSNL1, DLG4, DRD1 and PLEXNA2 were cloned into the 3′ UTR of a luciferase reporter gene construct and co-transfected into a recipient cell line with miRNA or anti-miRs (miRNA antagonists). The extent of reporter gene activity and the influence of miRNA were then determined by measuring the relative luciferase activity (FIG. 4c) (Lewis et al, Cell 115L787-798, 2003). Many of these constructs behaved in accordance with expectation and were significantly repressed in the presence of synthetic miRNA, and significantly de-repressed (increased luciferase) in the presence of the corresponding anti-miR (FIG. 4c). The most consistently responsive targets were derived from the 3′ UTR of RGS4, GRM7, GRIN3A and RELN, whereas, the least responsive was PLEXNA2. In respect to the miRNA, miR107 appeared to have the greatest overall effect, whereas miR-195 had the least effect on these target gene constructs. Collectively these reporter assays demonstrated a potential relationship between genes reported to be associated with schizophrenia and a large functionally-related group of up-regulated miRNA.

### Example 6

### Treatment of Schizophrenia in Animal Models

Antagonists specific for the miRNAs identified as being upregulated in schizophrenia (including hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e) are administered in the following animal models.

### Phencyclidine (PCP)/Ketamine Model—NMDA Receptor Antagonist Model of Schizophrenia

PCP and/or ketamine (as well as other NMDA receptor antagonists) administration to animals induces behaviours and biological effects that are similar to the symptoms of schizophrenia in humans. Following acute exposure or long-term exposure of animals (for example, rodents and non-human primates) to PCP, effects on one or more of the following is assessed:

frontal cortex function

temporal cortex function

sensorimotor gating

motor function

motivation

associative processes

social behaviour

locomotion

The effects of the hereinbefore-described antagonists on the above phenotypes are assessed by administering the antagonists to the PCP-treated animal.

### Dominant-Negative (DN) Disrupted-In-Schizophrenia-1 (DISC1) Mice

In this transgenic model, a dominant-negative form of DISC1 (DN-DISC1) is expressed under the αCaMKII promoter. DN-DISC1 mice have enlarged lateral ventricles particularly on the left side, suggesting a link to the asymmetrical change in anatomy found in brains of patients with schizophrenia. Furthermore, selective reduction in the immunoreactivity of parvalbumin in the cortex, a marker for an interneuron deficit that may underlie cortical asynchrony, is observed in the DN-DISC1 mice. DN-DISC1 mice also display several behavioral abnormalities, including hyperactivity, disturbance in sensorimotor gating and olfactory-associated behavior, and an anhedonia/depression-like deficit.

### Neonatal Brain Lesion Models

Neonatal damage of restricted brain regions of rodents or non-human primates disrupts development of the hippocampus, a brain area consistently implicated in human schizophrenia. The lesions involve regions of the hippocampus that directly project to the prefrontal cortex, i.e., ventral hippocampus and ventral subiculum, and that correspond to the anterior hippocampus in humans, a region that shows anatomical abnormalities in schizophrenia.

For example, neonatal excitotoxic lesions of the rat ventral hippocampus (VH) lead in adolescence or early adulthood to the emergence of abnormalities in a number of dopamine-related behaviors, which bear close resemblance to behaviors seen in animals sensitized to psychostimulants. In adolescence and adulthood (postnatal day 56 and older), rats with VH lesions display markedly changed behaviours thought to be primarily linked to increased mesolimbic/nigrostriatal dopamine transmission (motor hyperresponsiveness to stress and stimulants, enhanced stereotypies). They also show enhanced sensitivity to glutamate antagonists (MK-801 and PCP), deficits in PPI and latent inhibition, impaired social behaviors and working memory problems, phenomena showing many parallels with schizophrenia.

Such models as described above also allow for elucidation of a molecular signature associated with the various induced phenotypes, and allow for the molecular consequences of treatment with the hereinbefore antagonists to be investigated.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Beveridge et al, Hum Mol Genet 17:1156-1168, 2008
Bowden et al, BMC Genomics 9:199, 2008
Callicott et al, Am. J. Psychiatry. 160:709-719, 2003
Dennis et al, Genome Biol 4:P3, 2003
Ebert et al. Nature Methods 4(9):721-726, 2007
Gregory et al, Nature 432:235-240, 2004
Hakak et al, Proc Natl Acad Sci USA 98:4746-4751, 2001
Harrison Curr Opin Neurobiol 7:285-289, 1997
Igloi Anal Biochem 233:124-129, 1996
Kelly et al, Ir. J. Med. Sci. 172:37-40, 2003
Kim and Webster Correlation analysis between genome-wide expression profiles and cytoarchitectural abnormalities in the prefrontal cortex of psychiatric disorders 2008
Krutzfeldt et al. Nature 438:685-689, 2005
Lewis et al, Cell 115L787-798, 2003
Livak K J & Schmittgen T D Methods 25:402-408, 2001
Liu et al, Nucleic Acids Res 9:2811-2824, 2008
MacDonald et al, Arch. Gen. Psychiatry. 60:57-65, 2003
Mirnics et al, Neuron 28:53-67, 2000
Niendam et al, Am. J. Psychiatry. 160:2060-2062, 2003
Raymond et al, RNA 11:1737-1744, 2005
Sempere et al, Genome Biol 5:R13, 2004
Tepest et al, Biol, Psychiatry. 54:1234-1240, 2003
Thomson et al, Genes Dev 20:2202-2207, 2006
Thomson et al, Nat methods 1:47-53, 2004
Tusher et al, Proc Natl Acad Sci USA 98:5116-5121, 2001
U.S. Pat. No. 5,800,998
U.S. Pat. No. 5,837,492
U.S. Pat. No. 5,891,628
Weidenhofer et al, Mol Cell Neurosci 31:243-250, 2006
Winterer et al, Arch. Gen. Psychiatry. 60:1158-1167, 2003

What is claimed is:

1. A method for detecting a risk profile for schizophrenia or a manifestation thereof or a sub-threshold phenotype or state thereof in a subject, the method comprising identifying an elevation in expression of the DGCR8 gene or a homolog thereof or a genetic molecule associated therewith wherein an elevation in DGCR8 or its homolog or associated genetic molecule is indicative of a risk of having or developing symptoms of schizophrenia.

2. The method of claim 1 wherein the genetic molecule associated with DGCR8 is selected from hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2.

3. The method of claim 2 wherein the genetic molecule associated with DGCR8 is an miRNA.

4. The method of claim 3 wherein the genetic molecule associated with DGCR8 represents global miRNA expression.

5. The method of claim 3 wherein the miRNA is a member of the miR-15 or miR-107 family of miRNAs.

6. The method of claim 1 wherein the subject is human.

7. The method of claim 6 wherein expression of DGCR8 or its homolog or a genetic molecule associated therewith is the cerebral cortex including superior temporal gyrus or dorsolateral prefrontal cortex.

8. The method of claim 6 wherein the expression is DGCR8 or its homolog or a genetic material associated therewith is in a neural cell or neural fluid.

9. The method of claim 6 wherein the expression is DGCR8 or its homolog or a genetic material associated therewith is in a lymphocyte or other immune cells.

10. The method of claim 3 wherein an increased miRNA level results in down regulation of a gene which itself is an indicator of schizophrenia.

11. A method for stratifying subjects for schizophrenia, said method comprising determining levels of expression of DGCR8 or a homolog thereof or a genetic molecule associated therewith wherein an elevation in DGCR8 or its homolog or associated genetic molecule places a subject in a group of schizophrenia or at risk schizophrenia subjects.

12. The method of claim 11 wherein the genetic molecule associated with DGCR8 is selected from hsa-miR-107, hsa-miR-15a, hsa-miR-15b, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2.

13. The method of claim 12 wherein the genetic molecule associated with DGCR8 is an miRNA.

14. The method of claim 13 wherein the genetic molecule associated with DGCR8 represents global miRNA expression.

15. The method of claim 13 wherein the miRNA is a member of the miR-15 or miR-107 family of miRNAs.

16. The method of claim 11 wherein the subject is human.

17. The method of claim 16 wherein expression of DGCR8 or its homolog or a genetic molecule associated therewith is the cerebral cortex including superior temporal gyrus or dorsolateral prefrontal cortex.

18. The method of claim 16 wherein the expression is DGCR8 or its homolog or a genetic material associated therewith is in a neural cell or neural fluid.

19. The method of claim 16 wherein the expression is DGCR8 or its homolog or a genetic material associated therewith is in a lymphocyte or other immune cells.

20. The method of claim 13 wherein an increased miRNA level results in down regulation of a gene which itself is an indicator of schizophrenia.

21. A method for identifying a genetic basis behind diagnosing or treating schizophrenia or a manifestation thereof including a sub-threshold phenotype or state, the method comprising obtaining a biological sample from an individual and detecting the level of expression of DGCR8 or homolog thereof or a genetic molecule associated therewith wherein the presence of an elevated level of DGCR8 expression or an associated genetic molecule is instructive or predictive of schizophrenia or related conditions.

22. A method for treating schizophrenia or a manifestation thereof or a sub-threshold phenotype or state thereof in a subject, said method comprising administering to the subject a medicament which modulates the levels of a genetic indicator selected from the list comprising hsa-miR-107, hsa-miR-15a, hsa-miR-15b-R, hsa-miR-16, hsa-miR-128a, hsa-miR-181a, hsa-miR-181b, hsa-miR-181c, hsa-miR-195, hsa-miR-19a, hsa-miR-20a, hsa-miR-219, hsa-miR-26b, hsa-miR-27a, hsa-miR-29c, hsa-miR-328, hsa-miR-338, hsa-miR-7, hsa-miR-let-7d, hsa-miR-let-7e, FXR2, DICER, DGCR8, DROSHA, XPO5, DDX26, DDX5 and FXR2.

23. The method of claim 22, wherein the medicament is an antagonist selected from an antisense molecule, an antagomiR and a microRNAs sponge.

24. The method of claim 22 wherein the medicament targets DGCR8.

25. The method of claim 22 wherein the medicament targets a family member of miR-15 or miR-107.

26. The method of claim 22 wherein the subject is a human.


SEQUENCE LISTING (119 sequences)

SEQ ID NO: 1 - RNA, 22 nt, Artificial Sequence
  hsa-miR-15a oligonucleotide
uagcagcacauaaugguuugug

SEQ ID NO: 2 - RNA, 22 nt, Artificial Sequence
  hsa-miR-15b oligonucleotide
uagcagcacaucaugguuuaca

SEQ ID NO: 3 - RNA, 21 nt, Artificial Sequence
  hsa-miR-195 oligonucleotide
uagcagcacagaaauauuggc

SEQ ID NO: 4 - RNA, 22 nt, Artificial Sequence
  hsa-miR-16 oligonucleotide
uagcagcacguaaauauuggcg

SEQ ID NO: 5 - RNA, 23 nt, Artificial Sequence
  hsa-miR-107 oligonucleotide
agcagcacuguacagggcuauca

SEQ ID NO: 6 - RNA, 23 nt, Artificial Sequence
  3′-5′ HTR2A-107 MRE oligonucleotide
acuaucgggacauguuacgacga

SEQ ID NO: 7 - RNA, 24 nt, Artificial Sequence
  5′-3′ HTR2A-107 MRE oligonucleotide
uggaaaccuugcugcuaugcuguu

SEQ ID NO: 8 - DNA, 23 bp, Artificial Sequence
  U6-probe oligonucleotide
gccatgctaatcttctctgtatc

SEQ ID NO: 9 - DNA, 24 bp, Artificial Sequence
  U6-F339 oligonucleotide
cggcagcacatatactaaaattgg

SEQ ID NO: 10 - DNA, 22 bp, Artificial Sequence
  U49-F oligonucleotide
atcactaataggaagtgccgtc

SEQ ID NO: 11 - DNA, 21 bp, Artificial Sequence
  U49-R oligonucleotide
acaggagtagtcttcgtcagt

SEQ ID NO: 12 - DNA, 20 bp, Artificial Sequence
  U44-F oligonucleotide
tgatagcaaatgctgactga

SEQ ID NO: 13 - DNA, 22 bp, Artificial Sequence
  U44-R oligonucleotide
cagttagagctaattaagacct

SEQ ID NO: 14 - DNA, 15 bp, Artificial Sequence
  107-F oligonucleotide
agcagcattgtacag

SEQ ID NO: 15 - DNA, 25 bp, Artificial Sequence
  107-R oligonucleotide
gtaaaacgacggccagttgatagcc

SEQ ID NO: 16 - DNA, 13 bp, Artificial Sequence
  15a-Fb oligonucleotide
tagcagcacataa

SEQ ID NO: 17 - DNA, 26 bp, Artificial Sequence
  15a-R oligonucleotide
gtaaaacgacggccagtcacaaacca

SEQ ID NO: 18 - DNA, 14 bp, Artificial Sequence
  15b-F oligonucleotide
tagcagcacatcat

SEQ ID NO: 19 - DNA, 25 bp, Artificial Sequence
  15b-R oligonucleotide
gtaaaacgacggccagttgtaaacc

SEQ ID NO: 20 - DNA, 14 bp, Artificial Sequence
  16-F oligonucleotide
tagcagcacatcat

SEQ ID NO: 21 - DNA, 25 bp, Artificial Sequence
  16-R oligonucleotide
gtaaaacgacggccagttgtaaacc

SEQ ID NO: 22 - DNA, 13 bp, Artificial Sequence
  128a-F oligonucleotide
tcacagtgaaccg

SEQ ID NO: 23 - DNA, 26 bp, Artificial Sequence
  128a-R oligonucleotide
gtaaaacgacggccagtaaaagagac

SEQ ID NO: 24 - DNA, 14 bp, Artificial Sequence
  181a-F oligonucleotide
aacattcaacgctg

SEQ ID NO: 25 - DNA, 26 bp, Artificial Sequence
  181a-R oligonucleotide
gtaaaacgacggccagtactcaccga

SEQ ID NO: 26 - DNA, 18 bp, Artificial Sequence
  181b-F oligonucleotide
tttctaacattcattgct

SEQ ID NO: 27 - DNA, 18 bp, Artificial Sequence
  181b-R oligonucleotide
caaccttctcccaccgac

SEQ ID NO: 28 - DNA, 12 bp, Artificial Sequence
  195-F oligonucleotide
tagcagcacaga

SEQ ID NO: 29 - DNA, 26 bp, Artificial Sequence
  195-R oligonucleotide
gtaaaacgacggccagtgccaatatt

SEQ ID NO: 30 - DNA, 15 bp, Artificial Sequence
  19a-F oligonucleotide
tgtgcaaatctatgc

SEQ ID NO: 31 - DNA, 25 bp, Artificial Sequence
  19a-R oligonucleotide
gtaaaacgacggccagttcagtttt

SEQ ID NO: 32 - DNA, 16 bp, Artificial Sequence
  20a-F oligonucleotide
taaagtgcttatagtg

SEQ ID NO: 33 - DNA, 24 bp, Artificial Sequence
  20a-R oligonucleotide
gtaaaacgacggccagtctacctg

SEQ ID NO: 34 - DNA, 13 bp, Artificial Sequence
  219-F oligonucleotide
tgattgtccaaac

SEQ ID NO: 35 - DNA, 25 bp, Artificial Sequence
  219-F oligonucleotide
gtaaaacgacggccagtagaattgc

SEQ ID NO: 36 - DNA, 15 bp, Artificial Sequence
  26b-F oligonucleotide
ttcaagtaattcagg

SEQ ID NO: 37 - DNA, 24 bp, Artificial Sequence
  26b-R oligonucleotide
gtaaaacgacggccagtaacctat

SEQ ID NO: 38 - DNA, 13 bp, Artificial Sequence
  27a-F oligonucleotide
ttcacagtggcta

SEQ ID NO: 39 - DNA, 25 bp, Artificial Sequence
  27a-R oligonucleotide
gtaaaacgacggccagtgcggaact

SEQ ID NO: 40 - DNA, 14 bp, Artificial Sequence
  29b-F oligonucleotide
tagcaccatttgaa

SEQ ID NO: 41 - DNA, 25 bp, Artificial Sequence
  29c-R oligonucleotide
gtaaaacgacggccagttaaccgat

SEQ ID NO: 42 - DNA, 14 bp, Artificial Sequence
  338-F oligonucleotide
aacaatatcctggt

SEQ ID NO: 43 - DNA, 25 bp, Artificial Sequence
  338-R oligonucleotide
gtaaaacgacggccagtcactcagc

SEQ ID NO: 44 - DNA, 14 bp, Artificial Sequence
  7-F oligonucleotide
tggaagactagtga

SEQ ID NO: 45 - DNA, 26 bp, Artificial Sequence
  7-R oligonucleotide
gtaaaacgacggccagtacaacaaaa

SEQ ID NO: 46 - DNA, 14 bp, Artificial Sequence
  let-7d-F oligonucleotide
agaggtagtaggtt

SEQ ID NO: 47 - DNA, 25 bp, Artificial Sequence
  let-7d-R oligonucleotide
gtaaaacgacggccagtaactatgc

SEQ ID NO: 48 - DNA, 13 bp, Artificial Sequence
  let-7e-F oligonucleotide
tgaggtaggaggt

SEQ ID NO: 49 - DNA, 25 bp, Artificial Sequence
  let-7e-R oligonucleotide
gtaaaacgacggccagtactataca

SEQ ID NO: 50 - DNA, 17 bp, Artificial Sequence
  M13-F oligonucleotide
gtaaaacgacggccagt

SEQ ID NO: 51 - DNA, 21 bp, Artificial Sequence
  GUSB-F oligonucleotide
gccaatgaaaccaggtatccc

SEQ ID NO: 52 - DNA, 24 bp, Artificial Sequence
  GUSB-R oligonucleotide
gctcaagtaaacaggctgttttcc

SEQ ID NO: 53 - DNA, 20 bp, Artificial Sequence
  HMBS-F oligonucleotide
gagagtgattcgcgtgggta

SEQ ID NO: 54 - DNA, 20 bp, Artificial Sequence
  HMBS-R oligonucleotide
cagggtacgaggctttcaat

SEQ ID NO: 55 - DNA, 20 bp, Artificial Sequence
  FXR2-F oligonucleotide
accgccagccagtgactgtg

SEQ ID NO: 56 - DNA, 21 bp, Artificial Sequence
  FXR2-R oligonucleotide
agtcacccttctgtcctgaaa

SEQ ID NO: 57 - DNA, 21 bp, Artificial Sequence
  DICER1-F oligonucleotide
cacatcaatagatactgtgct

SEQ ID NO: 58 - DNA, 21 bp, Artificial Sequence
  DICER-R oligonucleotide
ttggtggaccaacaatggagg

SEQ ID NO: 59 - DNA, 18 bp, Artificial Sequence
  DGCR8-F oligonucleotide
gctgaggaaagggaggag

SEQ ID NO: 60 - DNA, 17 bp, Artificial Sequence
  DGCR8-R oligonucleotide
acgtccacggtgcacag

SEQ ID NO: 61 - DNA, 24 bp, Artificial Sequence
  DROSHA-F oligonucleotide
aagcgttaataggagctgtttact

SEQ ID NO: 62 - DNA, 21 bp, Artificial Sequence
  DROSHA-R oligonucleotide
cgtccaaataactgcttggct

SEQ ID NO: 63 - DNA, 19 bp, Artificial Sequence
  XPO5-F oligonucleotide
atatatgaggcactgcgcc

SEQ ID NO: 64 - DNA, 21 bp, Artificial Sequence
  XPO5-R oligonucleotide
aaactggtccagtgagtcctt

SEQ ID NO: 65 - DNA, 23 bp, Artificial Sequence
  DDX26-F2 oligonucleotide
agatccgaaagccaggaagaaaa

SEQ ID NO: 66 - DNA, 23 bp, Artificial Sequence
  DDX26-R2 oligonucleotide
tttgtaaactgccttgcacatgc

SEQ ID NO: 67 - DNA, 21 bp, Artificial Sequence
  DDX5-F oligonucleotide
aaggatgaaaaacttattcgt

SEQ ID NO: 68 - DNA, 21 bp, Artificial Sequence
  DDX5-R oligonucleotide
ttttccatgtttgaattcatt

SEQ ID NO: 69 - DNA, 21 bp, Artificial Sequence
  DDX17-F oligonucleotide
gtgaaaaagaccacaagttga

SEQ ID NO: 70 - DNA, 21 bp, Artificial Sequence
  DDX17-R oligonucleotide
tacacatagctggccaaccat

SEQ ID NO: 71 - DNA, 20 bp, Artificial Sequence
  FXR2-F oligonucleotide
accgccagccagtgactgtg

SEQ ID NO: 72 - DNA, 21 bp, Artificial Sequence
  FXR2-R oligonucleotide
agtcacccttctgtcctgaaa

SEQ ID NO: 73 - DNA, 20 bp, Artificial Sequence
  pri-181b-2-Fl oligonucleotide
aagaagagccaggagtcagc

SEQ ID NO: 74 - DNA, 20 bp, Artificial Sequence
  pri-181b-2-Rl oligonucleotide
tcagttggtggggttgcctt

SEQ ID NO: 75 - DNA, 20 bp, Artificial Sequence
  pre-181b-2-F oligonucleotide
ctgatggctgcactcaacat

SEQ ID NO: 76 - DNA, 23 bp, Artificial Sequence
  pre-181b-2-R oligonucleotide
tgatcagtgagttgattcagact

SEQ ID NO: 77 - DNA, 16 bp, Artificial Sequence
  pri-26b-F oligonucleotide
ccgtgctgtgctccct

SEQ ID NO: 78 - DNA, 22 bp, Artificial Sequence
  pri-26b-R oligonucleotide
cgagccaagtaatggagaacag

SEQ ID NO: 79 - DNA, 23 bp, Artificial Sequence
  pre-26b-F oligonucleotide
gacccagttcaagtaattcagga

SEQ ID NO: 80 - DNA, 22 bp, Artificial Sequence
  pre-26b-R oligonucleotide
cgagccaagtaatggagaacag

SEQ ID NO: 81 - DNA, 38 bp, Artificial Sequence
  VSNL1-107-T oligonucleotide
ctagttcctccaaagcctgggcagaaatgtgctgcaaa

SEQ ID NO: 82 - DNA, 38 bp, Artificial Sequence
  VSNL1-107-B oligonucleotide
agcttttgcagcacatttctgcccaggctttggaggaa

SEQ ID NO: 83 - DNA, 41 bp, Artificial Sequence
  RELN-107-T oligonucleotide
ctagtttacttgttatgttgtaatattttgctgctgaattt

SEQ ID NO: 84 - DNA, 41 bp, Artificial Sequence
  ReLN-107-B oligonucleotide
agctaaattcagcagcaaaatattacaacataacaagtaaa

SEQ ID NO: 85 - DNA, 41 bp, Artificial Sequence
  HTR2A-107-T oligonucleotide
ctagctattttcaagtggaaaccttgctgctatgctgttca

SEQ ID NO: 86 - DNA, 41 bp, Artificial Sequence
  HTR2A-107-B oligonucleotide
agcttgaacagcatagcagcaaggtttccacttgaaaatag

SEQ ID NO: 87 - DNA, 36 bp, Artificial Sequence
  GRIN3A-107-T oligonucleotide
ctaggcacaaaccctatcaagagctgctgcttccct

SEQ ID NO: 88 - DNA, 36 bp, Artificial Sequence
  GRIN3A-107-B oligonucleotide
agctagggaagcagcagctcttgatagggtttgtgc

SEQ ID NO: 89 - DNA, 38 bp, Artificial Sequence
  PLEXNA2-107-T oligonucleotide
ctaggacagttctgcctctgtgactgctgctttgcatg

SEQ ID NO: 90 - DNA, 38 bp, Artificial Sequence
  PLEXNA2-107-B oligonucleotide
agctcatgcaaagcagcagtcacagaggcagaactgtc

SEQ ID NO: 91 - DNA, 38 bp, Artificial Sequence
  DLG4-107-T oligonucleotide
ctaggtccgggagccagggaagactggaaatgctgccg

SEQ ID NO: 92 - DNA, 38 bp, Artificial Sequence
  DLG4-107-B oligonucleotide
agctcggcagcatttccagtcttccctggctcccggac

SEQ ID NO: 93 - DNA, 47 bp, Artificial Sequence
  DRD1-107-T oligonucleotide
ctagaatttacgatcttaggtggtaatgaaaagtatatgctgctttg

SEQ ID NO: 94 - DNA, 47 bp, Artificial Sequence
  DRD1-107-B oligonucleotide
agctcaaagcagcatatacttttcattaccacctaagatcgtaaatt

SEQ ID NO: 95 - DNA, 44 bp, Artificial Sequence
  GRM7-107-T oligonucleotide
ctaggtttgtaataagtactttcgttaatcttgctgcttatgtg

SEQ ID NO: 96 - DNA, 44 bp, Artificial Sequence
  GRM-107-B oligonucleotide
agctcacataagcagcaagattaacgaaagtacttattacaaac

SEQ ID NO: 97 - DNA, 58 bp, Artificial Sequence
  RGS4-107-T oligonucleotide
aatgcactagtccacattgtagcctaatattcatgctgcctgccatgaagcttaatgc

SEQ ID NO: 98 - DNA, 58 bp, Artificial Sequence
  RGS4-107-B oligonucleotide
gcattaagcttcatggcaggcagcatgaatattaggctacaatgtggactagtgcatt

SEQ ID NO: 99 - RNA, 23 nt, Artificial Sequence
  miR-107+ oligonucleotide
agcagcauuguacagggcuauca

SEQ ID NO: 100 - RNA, 23 nt, Artificial Sequence
  miR-107- oligonucleotide
auagcccuguacaaugcuguauu

SEQ ID NO: 101 - RNA, 22 nt, Artificial Sequence
  miR-15a+ oligonucleotide
uagcagcacauaaugguuugug

SEQ ID NO: 102 - RNA, 22 nt, Artificial Sequence
  miR-15a- oligonucleotide
caaaccauuaugugcuguuauu

SEQ ID NO: 103 - RNA, 22 nt, Artificial Sequence
  miR-15b+ oligonucleotide
uagcagcacaucaugguuuaca

SEQ ID NO: 104 - RNA, 22 nt, Artificial Sequence
  miR-15b- oligonucleotide
uaaaccaugaugugcuguuauu

SEQ ID NO: 105 - RNA, 22 nt, Artificial Sequence
  miR-16+ oligonucleotide
uagcagcacguaaauauuggcg

SEQ ID NO: 106 - RNA, 22 nt, Artificial Sequence
  miR-16- oligonucleotide
ccaauauuuacgugcuguuauu

SEQ ID NO: 107 - RNA, 21 nt, Artificial Sequence
  miR-195+ oligonucleotide
uagcagcacagaaauauuggc

SEQ ID NO: 108 - RNA, 21 nt, Artificial Sequence
  miR-195- oligonucleotide
caauauuucugugcuguuauu

SEQ ID NO: 109 - RNA, 22 nt, Artificial Sequence
  control-miRNA-1+ oligonucleotide
auccaccacguaaauauuggcg

SEQ ID NO: 110 - RNA, 22 nt, Artificial Sequence
  control-miRNA-1- oligonucleotide
ccaauauuuacgugguggaucg

SEQ ID NO: 111 - RNA, 23 nt, Artificial Sequence
  control-miRNA-2+ oligonucleotide
uccaccaauguacagggcuauca

SEQ ID NO: 112 - RNA, 23 nt, Artificial Sequence
  control-miRNA-2- oligonucleotide
auagcccuguacauuggugaauu

SEQ ID NO: 113 - DNA, 21 bp, Artificial Sequence
  anti-miR-107 oligonucleotide
tgatagccctgtacaatgctg

SEQ ID NO: 114 - DNA, 22 bp, Artificial Sequence
  anti-miR-15a oligonucleotide
cacaaaccattatgtgctgcta

SEQ ID NO: 115 - DNA, 22 bp, Artificial Sequence
  anti-miR-15b oligonucleotide
tgtaaaccatgatgtgctgcta

SEQ ID NO: 116 - DNA, 22 bp, Artificial Sequence
  anti-miR-16 oligonucleotide
cgccaatatttacgtgctgcta

SEQ ID NO: 117 - DNA, 21 bp, Artificial Sequence
  atni-miR-195 oligonucleotide
gccaatatttctgtgctgcta

SEQ ID NO: 118 - DNA, 22 bp, Artificial Sequence
  control-anti-miR-1 oligonucleotide
cgccaatatttacgtggtggat

SEQ ID NO: 119 - DNA, 21 bp, Artificial Sequence
  control-anti-miR-2 oligonucleotide
tgatagccctgtacattggtg